SEQUENCE LISTING (6 sequences)

SEQ ID NO: 1 - DNA, 1616 bp, Homo sapiens
gccaaggctggggcaggggagtcagcagaggcctcgctcgggcgcccagtggtcctgccgcctggtctcacctcgctatggttcgtctgcctctgcagtgcgtcctctggggctgcttgctgaccgctgtccatccagaaccacccactgcatgcagagaaaaacagtacctaataaacagtcagtgctgttctttgtgccagccaggacagaaactggtgagtgactgcacagagttcactgaaacggaatgccttccttgcggtgaaagcgaattcctagacacctggaacagagagacacactgccaccagcacaaatactgcgaccccaacctagggcttcgggtccagcagaagggcacctcagaaacagacaccatctgcacctgtgaagaaggctggcactgtacgagtgaggcctgtgagagctgtgtcctgcaccgctcatgctcgcccggctttggggtcaagcagattgctacaggggtttctgataccatctgcgagccctgcccagtcggcttcttctccaatgtgtcatctgctttcgaaaaatgtcacccttggacaagctgtgagaccaaagacctggttgtgcaacaggcaggcacaaacaagactgatgttgtctgtggtccccaggatcggctgagagccctggtggtgatccccatcatcttcgggatcctgtttgccatcctcttggtgctggtctttatcaaaaaggtggccaagaagccaaccaataaggccccccaccccaagcaggaaccccaggagatcaattttcccgacgatcttcctggctccaacactgctgctccagtgcaggagactttacatggatgccaaccggtcacccaggaggatggcaaagagagtcgcatctcagtgcaggagagacagtgaggctgcacccacccaggagtgtggccacgtgggcaaacaggcagttggccagagagcctggtgctgctgctgctgtggcgtgagggtgaggggctggcactgactgggcatagctccccgcttctgcctgcacccctgcagtttgagacaggagacctggcactggatgcagaaacagttcaccttgaagaacctctcacttcaccctggagcccatccagtctcccaacttgtattaaagacagaggcagaagtttggtggtggtggtgttggggtatggtttagtaatatccaccagaccttccgatccagcagtttggtgcccagagaggcatcatggtggcttccctgcgcccaggaagccatatacacagatgcccattgcagcattgtttgtgatagtgaacaactggaagctgcttaactgtccatcagcaggagactggctaaataaaattagaatatatttatacaacagaatctcaaaaacactgttgagtaaggaaaaaaaggcatgctgctgaatgatgggtatggaactttttaaaaaagtacatgcttttatgtatgtatattgcctatggatatatgtataaatacaatatgcatcatatattgatataacaagggttctggaagggtacacagaaaacccacagctcgaagagtggtgacgtctggggtggggaagaagggtctggggg

SEQ ID NO: 2 - DNA, 2757 bp, Homo sapiens
gacaagtactgagtgaactcaaaccctctgtaaagtaacagaagttagaaggggaaatgtcgcctctctgaagattacccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtcccacctccatttgcaattgacctcttctgggaacttcctcagatggacaagattaccccaccttgccctttacgtatctgctcttaggtgcttcttcacttcagttgctttgcaggaagtgtctagaggaatatggtgggcacagaagtagctctggtgaccttgatcaaggtgttttgaaatgcagaattcttgagttctggaagggactttagagaataccagtgttattaatgacaaaggcactgaggcccagggaggtgacccgaattataaaggccagcgccagaacccagatttcctaactctggtgctctttccctttatcagtttgactgtggcctgttaactggtatatacatatatatgtcaggcaaagtgctgctggaagtagaatttgtccaataacaggtcaacttcagagactatctgatttcctaatgtcagagtagaagattttatgctgctgtttacaaaagcccaatgtaatgcataggaagtatggcatgaacatctttaggagactaatggaaatattattggtgtttacccagtattccatttttttcattgtgttctctattgctgctctctcactcccccatgaggtacagcagaaaggagaactatccaaaactaatttcctctgacatgtaagacgaatgatttaggtacgtcaaagcagtagtcaaggaggaaagggatagtccaaagacttaactggttcatattggactgataatctctttaaatggctttatgctagtttgacctcatttgtaaaatatttatgagaaagttctcatttaaaatgagatcgttgtttacagtgtatgtactaagcagtaagctatcttcaaatgtctaaggtagtaactttccatagggcctccttagatccctaagatggctttttctccttggtatttctgggtctttctgacatcagcagagaactggaaagacatagccaactgctgttcatgttactcatgactcctttctctaaaactgccttccacaattcactagaccagaagtggacgcaacttaagctgggataatcacattatcatctgaaaatctggagttgaacagcaaaagaagacaacatttctcaaatgcacatctcatggcagctaagccacatggctgggatttaaagcctttagagccagcccatggctttagctacctcactatgctgcttcacaaaccttgctcctgtgtaaaactatattctcagtgtagggcagagaggtctaacaccaacataaggtactagcagtgtttcccgtattgacaggaatacttaactcaataattcttttcttttccatttagtaacagttgtgatgactatgtttctattctaagtaattcctgtattctacagcagatactttgtcagcaatactaagggaagaaacaaagttgaaccgtttctttaataa

SEQ ID NO: 3 - DNA, 2399 bp, Homo sapiens
agtcattgccgaggaaggcttgcacagggtgaaagctttgcttctctgctgctgtaacagggactagcacagacacacggatgagtggggtcatttccagatattaggtcacagcagaagcagccaaaatggatccccagtgcactatgggactgagtaacattctctttgtgatggccttcctgctctctgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgttttttttttaaatagacctctcaatttctggaaaactgccttttatctgcccagaattctaagctggtgccccactgaattttgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcatttaaaaaaaaaaaaaa

SEQ ID NO: 4 - DNA, 31 bp, Artificial Sequence
  Antisense oligodeoxynucleotide
cacagccgaggcaaagacaccatgcagggca

SEQ ID NO: 5 - DNA, 29 bp, Artificial Sequence
  Antisense oligodeoxynucleotide
gggaaagccaggaatctagagccaatgga

SEQ ID NO: 6 - DNA, 30 bp, Artificial Sequence
  Antisense oligodeoxynucleotide
tgggtgcttccgtaagttctggaacacgtc

(19) United States
(12) Patent Application Publication
(10) Pub. No.: US 2016/0230174 A1
(43) Pub. Date: Aug. 11, 2016

(54) TOLEROGENIC DENDRITIC CELLS TO TREAT INFLAMMATORY BOWEL DISEASE

(71) Applicant: UNIVERSITY OF PITTSBURGH-OF THE COMMONWEALTH SYSTEM OF HIGHER EDUCATION, Pittsburgh, PA (US)

(72) Inventors: Nick Giannoukakis, Coraopolis, PA (US); Massimo M. Trucco, Pittsburgh, PA (US)

(73) Assignee: University of Pittsburgh - Of the Commonwealth System of Higher Education, Pittsburgh, PA (US)

(21) Appl. No.: 15/025,208

(22) PCT Filed: Sep. 26, 2014
(86) PCT No.: PCT/US2014/057627

§ 371 Date: Mar. 25, 2016

Related U.S. Application Data
(60) Provisional application No. 61/883,106, filed on Sep. 26, 2013.

(51) Int. Cl. C12N 15/113; A61K 45/06; A61K 31/7088; A61K 35/15

(52) U.S. Cl. CPC C12N 15/1138; A61K 35/15; A61K 45/06; A61K 31/7088; C12N 2310/11; C12N 2320/30; C12N 2320/31; C12N 2320/32; C12N 2310/315

(57) ABSTRACT

Methods are disclosed herein for treating or preventing an inflammatory bowel disease in a subject. These methods include administering to a subject an effective amount of tolerogenic dendritic cells, wherein the tolerogenic dendritic cells comprise at least one of an antisense compound specific for CD40, and antisense compound specific for CD80 and an antisense compound specific for CD86.

### CROSS REFERENCE TO RELATED APPLICATIONS

This claims the benefit of U.S. Application No. 61/883,106, filed Sep. 26, 2013, which is incorporated by reference herein.

### FIELD

This relates to the treatment of inflammatory bowel disease, specifically to the use of antisense compounds for CD40, CD80 and/or CD86 to produce tolerogenic dendritic cells and to treat inflammatory bowel disease.

### BACKGROUND

Inflammatory Bowel Disease (IBD) includes Crohn's Disease (CD) and ulcerative colitis (UC); these conditions are chronic, inflammatory diseases of the gastrointestinal tract. While the clinical features vary somewhat between these two disorders, both are characterized by abdominal pain, diarrhea (often bloody), a variable group of extra-intestinal manifestations (including arthritis, uveitis, and skin changes) and the accumulation of inflammatory cells within the small intestine and colon (observed in pathologic biopsy or surgical specimens). Recent studies have identified variations in specific genes, including ATG16L1, interleukin (IL)23R, IRGM, and NOD2, that influence the risk of developing CD. As many as thirty human genes have been identified which contribute to ulcerative colitis susceptibility.

IBD affects both children and adults, and has a bimodal age distribution (one peak around 20, and a second around 40). IBD is a chronic, lifelong disease, and is considered an autoimmune disorder. IBD is found almost exclusively in the industrialized world. In the United States, IBD is the second most common autoimmune disease: there is an overall incidence of greater than 1 in 100,000 people. In addition, there is a clear trend towards an increasing incidence of IBD in both United States and Europe, particularly for CD.

There currently are a variety of treatments for IBD is varied. First line therapy typically includes salicylate derivatives, which are given orally or rectally. Response rates in uncomplicated CD are approximately 40% (compared to 20% for placebo). Corticosteroids are commonly used in the treatment of patients with more “refractory” disease, despite the side-effects. Additional treatment options include anti-metabolites (e.g., methotrexate, 6-mercaptopurine) and immunomodulators (such as antibodies that specifically bind the tumor necrosis factor (TNF)-α receptor). However, IBD remains difficult to diagnose and treat effectively. There is a clear need for improved methods for treating inflammatory bowel diseases.

### SUMMARY

Methods are disclosed herein for treating or preventing an IBD in a subject. The methods include administering to a subject an effective amount at least one of an antisense compound specific for CD40, and antisense compound specific for CD80 and an antisense compound specific for CD86. In some embodiments, the methods include administering to a subject an effective amount of an antisense compound specific for CD40, and antisense compound specific for CD80 and an antisense compound specific for CD86.

In some embodiments, the methods include administering to a subject an effective amount of tolerogenic dendritic cells, wherein the tolerogenic dendritic cells include at least one of an antisense compound specific for CD40, and antisense compound specific for CD80 and an antisense compound specific for CD86. In some embodiments, the IBD is CD or ulcerative colitis (UC). In additional embodiments, the method can include producing the tolerogenic dendritic cells. In further embodiments, the subject is human. In yet other embodiments, the dendritic cells are autologous.

In some non-limiting examples, the tolerogenic dendritic cells are autologous. In further non-limiting examples, tolerogenic dendritic cells include all of an antisense compound specific for CD40, and antisense compound specific for CD80 and an antisense compound specific for CD86.

The foregoing and other features and advantages of the disclosure will become more apparent from the following detailed description, which proceeds with reference to the accompanying figures.

### BRIEF DESCRIPTION OF THE DRAWINGS

FIGS. 1A-1B. Pre-treatment with cDC and iDC followed by a second injection of DC three days following induction of colitis with DSS attenuates weight loss in mice. FIG. 1A. The graph shows the median weight (solid symbols) of DSS-exposed mice that were injected with cDC, iDC or PBS vehicle as control three days prior to DSS exposure and then a second DC (or PBS vehicle) injection three days following DSS exposure. The bars represent the SD of n=4 mice. The graph represents the outcome of one of two mouse cohorts where each treatment group consisted of four mice. The outcomes of the DC treatments in both cohorts are identical. At each time point, the difference between the cDC/iDC medians and the control mouse median was statistically-significant (p<0.01, Kruskal-Wallis test). cDC indicates treatment with control DC generated in GM-CSF and IL-4 from bone marrow progenitors and iDC indicates treatment with DC generated in the presence of the antisense DNA oligonculeotides. FIG. 1B. The graph summarizes the % of starting weight (prior to DSS exposure and three days following the first DC injection) of the first cohort of treated mice which was reproducible in the second cohort (n=4 mice per treatment group). Neg Con indicates mice that received PBS vehicle and exposed to DSS, Colitis Con DC treated refers to mice exposed to DSS and treated with control DC while Colitis ASODN DC Treated refers to mice exposed to DSS and treated with the DC generated in the presence of the antisense oligodeoxyribonucleotides (ASODN). The differences between the median % starting weights of the cDC/iDC and those of the DSS-treated controls who exhibited severe colitis was significant (p<0.05, Mann-Whitney U-test).

FIGS. 2A-2B. Increased frequency in the spleen and the mesenteric lymph nodes of cDC and iDC-treated mice exposed to DSS. FIG. 2A. The figure outlines the gating strategy for the FACS analysis to measure CD4+CD25+Foxp3+Tregs. The data are representative of the measurements in the spleens of four mice of all treatment groups (DSS: DSS exposure alone; DSS+cDC; cDC pretreatment prior to DSS and then a second injection three days later; DSS+iDC: iDC pretreatment prior to DSS and then a second injection three days later; and control: no DSS exposure, injection of PBS vehicle. Quadrant 2-3 of the bottom panels represents the channels in which CD25+Foxp3+ cells were measured after gating for CD4 positivity (middle panels). FIG. 2B. The graph summarizes the frequency of Foxp3+Tregs in the spleens and mesenteric lymph nodes of DSS-exposed mice alone (No DC); DSS-exposed and cDC-injected mice (Control DC); DSS-exposed and iDC-injected mice (ASODN DC) and untreated control mice (No colitis). The bars represent the means of Foxp3+Tregs as a % of total cells (splenocytes or lymph node cells) and the error bars the SEM. For both spleen and lymph nodes, the difference in the means between the cDC/iDC and control mice (DSS alone or untreated) were statistically-significant (P<0.01, ANOVA).

FIGS. 3A-3B. Increased frequency of B10 Bregs in the mesenteric lymph nodes of cDC and iDC-treated mice exposed to DSS. FIG. 3A. The graph outlines the gating strategy for the FACS analysis to measure B220+CD19+CD11c-IL-10+CD1d+CD5+ B-cells (the B10 Bregs). The data are representative of the measurements in the mesenteric lymph nodes in four mice of all treatment groups (control: no DSS exposure, injection of PBS vehicle; DSS: DSS exposure alone; DSS+cDC; cDC pretreatment prior to DSS and then a second injection three days later; DSS+iDC: iDC pretreatment prior to DSS and then a second injection three days later. Quadrant 2-15 of the bottom panels represents the channels in which CD1d+CD5+ cells were measured after gating for sequential B220+CD19+ positivity, CD11c negativity and then IL-10 positivity (top and middle panels). FIG. 3B. The graph summarizes the frequency of B10 Bregs in the mesenteric lymph nodes of DSS-exposed mice alone (colitis); DSS-exposed and cDC-injected mice (colitis+DC); DSS-exposed and iDC-injected mice (colitis+iDC) and untreated control mice (no colitis). The bars represent the means of CD1d+CD5+IL-10+B220+CD19+CD11c-cells as a % of B220+CD19+ B-cells and the error bars the SEM. The difference in the means between the cDC/iDC and control mice (DSS alone or untreated) were statistically-significant (P<0.05, ANOVA).

FIGS. 4A-4B. Increased frequency of retinoic acid-producing DC in the spleen and mesenteric lymph nodes of cDC and iDC-treated mice exposed to DSS. FIG. 4A. The figure outlines the gating strategy for the FACS analysis to measure CD103+ or CD11c+ cells that produce retinoic acid (i.e. that are reactive with the ALDEFLUOR® reagent; ALDEFLUOR®+ cells). The data are representative of the measurements in the spleen and mesenteric lymph nodes in four mice of all treatment groups (DSS: DSS exposure alone; DSS+cDC; cDC pretreatment prior to DSS and then a second injection three days later; DSS+iDC: iDC pretreatment prior to DSS and then a second injection three days later. Quadrant 2-1 of the middle panels represents the channels in which CD103+ALDEFLUOR®+ cells were measured and Quadrant 2-5 represents the channels in which CD11c+ALDEFLUOR®+ cells were measured. FIG. 4B. The graph summarizes the frequency of CD11c+ALDEFLUOR®+ cells in the spleen and mesenteric lymph nodes as well as the CD103+ALDEFLUOR®+ cells in the spleens of DSS-exposed mice alone (colitis); DSS-exposed and cDC-injected mice (colitis+DC); and DSS-exposed and iDC-injected mice (colitis+iDC). CD103+ cells were detectable only in spleens of even untreated mice and not in the mesenteric lymph nodes. ALDH+ indicates ALDEFLUOR®-reactive cells. The bars represent the means of the double-positive cells as a % of total splenic and mesenteric lymph node cells and the error bars the SEM. The difference in the means between the cDC/iDC and control mice (DSS-exposed) were statistically-significant (P<0.01, ANOVA).

FIGS. 5A-5B. iDC treatment preferentially-attenuates colon inflammation of DSS-exposed mice. FIG. 5A. H&E staining of colons resected from DSS-exposed mice treated with cDC or iDC. Representative sections are shown at two magnifications (×5 and ×20). Untreated, DSS-exposed mice exhibit inflammatory as well as significant tissue architecture disruption. Even though cDC treatment does not prevent inflammatory foci formation, the architecture of the tissue remains intact. iDC treatment significantly-attenuates inflammation and preserves tissue architecture. FIG. 5B. Colitis inflammation in excised colons of 4 mice per treatment group (colitis: DSS exposure alone; cDC: control DC treatment with DSS exposure; and iDC: antisense oligonucleotide-generated DC treatment with DSS exposure) was scored in a blinded manner. Histologic scores were as follows: 0, normal; 1, ulcer or cell infiltration limited to the mucosa; 2, ulcer or limited cell infiltration in the submucosa; 3, focal ulcer involving all layers of the colon; 4, multiple lesions involving all layers of the colon, or necrotizing ulcer larger than 3 mm in length. The bars in the graph represent the mean score of all colon sections assessed and the error bars the SEM. The differences in scores between the cDC/iDC and control (DSS colitis) mouse colons were statistically-significant (p<0.05, MANOVA).

FIG. 6. Significant accumulation of Foxp3+ cells inside iDC-treated DSS-exposed mice as well as evidence of B-cell accumulation. The top panels show colon sections from DSS-exposed mice with or without DC administration (cDC/iDC) stained with antibodies specific for CD25 and Foxp3. Sections from iDC recipients exhibit substantial and widespread Foxp3 immunoreactivity. CD25 was not readily discernible. The bottom three panels are parallel sections stained with antibodies specific for CD19, IL-10 and retinoic acid receptor alpha (all isoforms). CD19 immunoreactivity is discernible lining the villi and the crypts. Retinoic acid receptor alpha expression is widespread in the colon villi, crypts and submucosa. The immunofluorescence microscopy was conducted at ×40 magnification.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand when appropriate. The Sequence Listing is submitted as an ASCII text file [90797-02_Sequence.txt, Sep. 26, 2014, 9.77 KB], which is incorporated by reference herein.

### DETAILED DESCRIPTION

Methods are disclosed herein for treating or preventing an IBD in a subject. The methods include administering to a subject an effective amount at least one of an antisense compound specific for CD40, and antisense compound specific for CD80 and an antisense compound specific for CD86, thereby treating or preventing the IBD. In some embodiments, the methods include administering to a subject an effective amount of an antisense compound specific for CD40, and antisense compound specific for CD80 and an antisense compound specific for CD86. These methods can include administering to a subject an effective amount of tolerogenic dendritic cells, wherein the tolerogenic dendritic cells include at least one of an antisense compound specific for CD40, and antisense compound specific for CD80 and an antisense compound specific for CD86. In some embodiments, the IBD is CD or UC.

### TERMS

Unless otherwise noted, technical terms are used according to conventional usage. Definitions of common terms in molecular biology may be found in Benjamin Lewin, Genes IX, published by Jones and Bartlett Publishers, 2007 (ISBN 0763740632); Kendrew et al. (eds.), The Encyclopedia of Molecular Biology, published by Blackwell Science Inc., 1998; and Robert A. Meyers (ed.), Molecular Biology and Biotechnology: a Comprehensive Desk Reference, published by VCH Publishers, Inc., 1995 (ISBN 1-56081-569-8). It is further to be understood that all base sizes or amino acid sizes, and all molecular weight or molecular mass values, given for nucleic acids or polypeptides are approximate, and are provided for description. The singular terms “a,” “an,” and “the” include plural referents unless context clearly indicates otherwise. Similarly, “A or B” is intended to indicate “A,” “B,” and “A and B,” unless the context clearly indicates otherwise. The term “comprises” means “includes.” Although methods and materials similar or equivalent to those described herein can be used in the practice or testing of the present disclosure, suitable methods and materials are described below. In addition, all GENBANK® accession numbers are herein incorporated by reference as they appear in the database. In case of conflict, the present specification, including explanations of terms, will control. In order to facilitate review of the various embodiments of the disclosure, the following explanations of specific terms are provided:

Administration: To provide or give a subject an agent, such as a therapeutic agent, by any effective route. Exemplary routes of administration include, but are not limited to, injection (such as subcutaneous, intramuscular, intradermal, intraperitoneal, and intravenous), oral, intraductal, sublingual, rectal, transdermal, intranasal, and inhalation routes.

Agent: Any protein, nucleic acid molecule (including chemically modified nucleic acids), compound, small molecule, organic compound, inorganic compound, microsphere or other molecule of interest. An agent can include a therapeutic agent, a diagnostic agent or a pharmaceutical agent. A therapeutic or pharmaceutical agent is one that alone or together with an additional compound induces the desired response (such as inducing a therapeutic or prophylactic effect when administered to a subject), including inhibiting or treating an IBD. For example, a “therapeutic agent” is a chemical compound, small molecule, or other composition, such as an antisense compound, antibody, protease inhibitor, hormone, microsphere, chemokine or cytokine, capable of inducing a desired therapeutic or prophylactic effect when properly administered to a subject.

Allogeneic and Autologous: Organisms, cells, tissues, organs, and the like from, or derived from, individuals of the same species, but wherein the organisms, cells, tissues, organs, and the like are genetically different one from another are “allogeneic.” Organisms, cells, tissues, organs, and the like from, or derived from, a single individual, or from a genetically identical individual are “autologous.” “Transplant rejection” refers to a partial or complete destruction of a transplanted cell, tissue, organ, or the like on or in a recipient of said transplant due to an immune response to an allogeneic cell or tissue.

Alteration in expression: An alteration in expression of a CD40, CD80 or CD86 gene product refers to a change or difference, such as an increase or decrease, in the level of the CD40, CD80 or CD86 gene product that is detectable in a biological sample relative to a control. An “alteration” in expression includes an increase in expression (up-regulation) or a decrease in expression (down-regulation). In some examples, an alteration in expression includes a change or difference, such as an increase or decrease, in the conversion of the information encoded in a gene into the gene product. In some examples, the difference is relative to a control or reference value, such as an amount of expression in a sample from a control subject.

Antisense, Sense, and Antigene: Double-stranded DNA (dsDNA) has two strands, a 5′->3′ strand, referred to as the plus strand, and a 3′->5′ strand, referred to as the minus strand. Because RNA polymerase adds nucleic acids in a 5′->3′ direction, the minus strand of the DNA serves as the template for the RNA during transcription. Thus, the RNA formed will have a sequence complementary to the minus strand, and identical to the plus strand (except that the base uracil is substituted for thymine).

Antisense compounds are compounds that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a DNA target.

Antisense compound: An oligomeric compound that is at least partially complementary to the region of a target nucleic acid molecule (such as CD40, CD80 or CD86) to which it hybridizes. As used herein, an antisense compound that is “specific for” a target nucleic acid molecule is one which specifically hybridizes with and alters expression of the target nucleic acid molecule and not other unrelated nucleic acid molecules. As used herein, a “target” nucleic acid is a nucleic acid molecule to which an antisense compound is designed to specifically hybridize and modulate expression.

Non-limiting examples of antisense compounds include primers, probes, antisense oligonucleotides, small inhibitory RNAs (siRNAs), micro RNAs (miRNAs), short hairpin RNAs (shRNAs) and ribozymes. As such, these compounds can be introduced as single-stranded, double-stranded, circular, branched or hairpin compounds and can contain structural elements such as internal or terminal bulges or loops. Double-stranded antisense compounds can be two strands hybridized to form double-stranded compounds or a single strand with sufficient self-complementarity to allow for hybridization and formation of a fully or partially double-stranded compound. In particular examples herein, the antisense compound is an antisense oligonucleotide, siRNA or ribozyme specific for CD40, CD80 or CD86.

In some examples, an antisense compound is an “antisense oligonucleotide.” An antisense oligonucleotide is a single-stranded antisense compound that is a nucleic acid-based oligomer specific for a target sequence of interest. An antisense oligonucleotide can include one or more chemical modifications to the sugar, base, and/or internucleoside linkages. Generally, antisense oligonucleotides are “DNA-like” such that when the antisense oligonucleotide hybridizes to a target RNA molecule, the duplex is recognized by RNase H (an enzyme that recognizes DNA:RNA duplexes), resulting in cleavage of the RNA.

Binding or stable binding: An oligonucleotide binds or stably binds to a target nucleic acid if a sufficient amount of the oligonucleotide forms base pairs or is hybridized to its target nucleic acid, to permit detection of that binding. Binding can be detected by either physical or functional properties of the target:oligonucleotide complex. Binding between a target and an oligonucleotide can be detected by any procedure known to one skilled in the art, including both functional or physical binding assays. Binding may be detected functionally by determining whether binding has an observable effect upon a biosynthetic process such as expression of a gene, DNA replication, transcription, translation and the like.

Physical methods of detecting the binding of complementary strands of DNA or RNA are well known in the art, and include such methods as DNase I or chemical footprinting, gel shift and affinity cleavage assays, Southern blotting, Northern blotting, dot blotting and light absorption detection procedures. For example, a method which is widely used, because it is so simple and reliable, involves observing a change in light absorption of a solution containing an oligonucleotide (or an analog) and a target nucleic acid at 220 to 300 nm as the temperature is slowly increased. If the oligonucleotide or analog has bound to its target, there is a sudden increase in absorption at a characteristic temperature as the oligonucleotide (or analog) and target dissociate or melt.

The binding between an oligomer and its target nucleic acid is frequently characterized by the temperature (Tₘ) at which 50% of the oligomer is melted from its target. A higher (Tₘ) means a stronger or more stable complex relative to a complex with a lower (Tₘ).

CD40: A member of the TNF-receptor superfamily that has been found to be essential in mediating a broad variety of immune and inflammatory responses including T cell-dependent immunoglobulin class switching, memory B cell development, and germinal center formation. An exemplary protein and nucleic acid sequence for human CD40 can be found as GENBANK® Accession No. NM_001250, Sep. 2, 2013, which is incorporated herein by reference.

CD80 (B7-1): A protein found on activated B cells and monocytes that provides a costimulatory signal necessary for T cell activation and survival. It is the ligand for two different proteins on the T cell surface: CD28 (for autoregulation and intercellular association) and CTLA-4 (for attenuation of regulation and cellular disassociation). CD80 works in tandem with CD86 to prime T cells. An exemplary protein and nucleic acid sequence for human CD40 can be found as GENBANK® Accession No. NM_005191.3, Sep. 2, 2013, which is incorporated herein by reference.

CD86 (B7-2): A protein that is a member of the immunoglobulin superfamily expressed on antigen-presenting cells that provides costimulatory signals necessary for T cell activation and survival. It is the ligand for two different proteins on the T cell surface: CD28 (for autoregulation and intercellular association) and CTLA-4 (for attenuation of regulation and cellular disassociation). CD86 works in tandem with CD80 to prime T cells. An exemplary protein and nucleic acid sequence for human CD40 can be found as GENBANK® Accession No. NM_001206924, Sep. 2, 2013, which is incorporated herein by reference.

Contacting: Placement in direct physical association, including both a solid and liquid form. Contacting an agent with a cell can occur in vitro by adding the agent to isolated cells or in vivo by administering the agent to a subject.

Decrease or downregulate: To reduce the quality, amount, or strength of something. In one example, a therapy decreases a sign or symptom of an IBD, such as Crone's disease or ulcerative colitis, in a subject, for example as compared to the response in the absence of the therapy.

In some examples, when used in reference to the expression of nucleic acid molecules (such as a mRNA), a reduction or downregulation refers to any process which results in a decrease in production of a gene product. Gene downregulation includes any detectable decrease in the production of a mRNA. In certain examples, production of a mRNA decreases by at least 2-fold, at least 3-fold, at least 4-fold, at least 5-fold, at least 6-fold, at least 8-fold, at least 10-fold, at least 15-fold, at least 20-fold, at least 30-fold or at least 40-fold, as compared to a control.

Dendritic cells (DCs): Antigen-presenting immune cells that process antigenic material and present it to other cells of the immune system, most notably to T cells but also to B cells. Immature DCs function to capture and process antigens. When DCs endocytose antigens, they process the antigens into smaller fragments, generally peptides, that are displayed on the DC surface, where they are presented to, for example, antigen-specific immune cells. After uptake of antigens, DCs migrate to the lymph nodes. Immature dendritic cells are characterized by high endocytic and micropinocytotic function. During maturation, DCs can be prompted by various signals, including signaling through Toll-like receptors (TLR), to express co-stimulatory signals that induce B cells or cognate effector T cells (Teff) to become activated and to proliferate, thereby initiating a B cell or T cell mediated immune response to the antigen. Alternatively, DCs can present antigen to immune cells without providing co-stimulatory signals (or while providing co-inhibitory signals), such that the immune cells are not properly activated. Such presentation can cause, for example, death or anergy of the immune cells recognizing the antigen, or can induce the generation and/or expansion of regulatory cells (Tregs or Bregs). The term “dendritic cells” includes differentiated dendritic cells, immature, and mature dendritic cells. These cells can be characterized by expression of certain cell surface markers (e.g., CD11c, MHC class II, and at least low levels of CD80 and CD86), CD11b, CD304 (BDCA4)). In some embodiments, DCs express CD8, CD103, CD1d, etc. Other DCs can be identified by the absence of lineage markers such as CD3, CD14, CD19, CD56, etc. In addition, dendritic cells can be characterized functionally by their capacity to stimulate alloresponses and mixed lymphocyte reactions (MLR).

Tolerogenic DCs” or “induced tolerogenic dendritic cells” refers to dendritic cells capable of suppressing immune responses or generating tolerogenic immune responses, such as polyclonal or antigen-specific regulatory T-cells and/or B-cells or suppressive T cell-mediated immune responses. Tolerogenic DCs can be characterized by specific tolerogenic immune response induction ex vivo and/or in vivo. In embodiments, induced tolerogenic dendritic cells have a tolerogenic phenotype that is characterized by at least one, if not all, of the following properties i) capable of converting naive T cells to Foxp3+T regulatory cells ex vivo and/or in vivo (e.g., inducing expression of FoxP3 in the naive T cells); blocking the conversion of naive T-cells to TH17 T-cells; iii) capable of deleting effector T cells ex vivo and/or in vivo; iv) retain their tolerogenic phenotype upon stimulation with at least one Toll-like receptor (TLR) agonist ex vivo (and, in some embodiments, increase expression of costimulatory molecules in response to such stimulus); and/or v) do not transiently increase their oxygen consumption rate upon stimulation with at least one TLR agonist ex vivo; and/or vi) capable of converting B cells to regulatory B cells ex vivo and/or in vivo.

Starting populations of cells comprising dendritic cells and/or dendritic cell precursors may be “induced” by treatment, for example, ex vivo to become tolerogenic. In some embodiments, starting populations of dendritic cells or dendritic cell precursors are differentiated into dendritic cells prior to, as part of, or after induction, for example by treatment with antisense compounds specific for CD40, CD80 and/or CD86. In some embodiments, induced dendritic cells comprise fully differentiated dendritic cells. In some embodiments, induced dendritic cells comprise both immature and mature dendritic cells. In some embodiments, induced dendritic cells are enriched for mature dendritic cells.

Determining or detecting the level of expression of a gene product: Detection of a level of expression in either a qualitative or quantitative manner, for example by detecting nucleic acid molecules or proteins, for instance using routine methods known in the art.

Effective amount: An amount of agent that is sufficient to generate a desired response, such as reducing or inhibiting one or more signs or symptoms associated with a condition or disease. When administered to a subject, a dosage will generally be used that will achieve target tissue concentrations. In some examples, an “effective amount” is one that treats one or more symptoms and/or underlying causes of any of a disorder or disease, such as an IBD. In some examples, an “effective amount” is a therapeutically effective amount in which the agent alone with an additional therapeutic agent(s), induces the desired response, such as a decrease in symptoms of the IBD or a decrease in inflammation of the digestive tract.

Inflammatory bowel diseases (IBD): Disease characterized by chronic, relapsing intestinal inflammation of obscure origin. In patients with IBD, ulcers and inflammation of the inner lining of the intestines lead to symptoms of abdominal pain, diarrhea, and rectal bleeding. There are two primary types of IBD, Crohn's disease (CD) and ulcerative colitis (UC); both of these diseases appear to result from the unrestrained activation of an inflammatory response in the intestine. However, collagenous colitis lymphocytic colitis, ischemic colitis, diversion colitis, Behcet's disease, and indeterminate colitis are also considered to be inflammatory bowel diseases.

The main difference between CD and UC is the location and nature of the inflammatory changes. CD can affect any part of the gastrointestinal tract, from mouth to anus (skip lesions), although a majority of the cases start in the terminal ileum. Ulcerative colitis, in contrast, is restricted to the colon and the rectum. Symptoms of IBD most commonly include fever, vomiting, diarrhea, bloody stool (hematochezia), abdominal pain, and weight loss, but also may include a host of other problems. The severity of symptoms may impair the quality of life of patients that suffer from IBD. For most patients, IBD is a chronic condition with symptoms lasting for months to years. It is most common in young adults, but can occur at any age. IBD especially common in people of Jewish descent and has racial differences in incidence as well.

Diagnosis of IBD can be based on the clinical symptoms or the use of a barium enema, but direct visualization (sigmoidoscopy or colonoscopy) is the most accurate test. Protracted IBD is a risk factor for colon cancer, and treatment of IBD can involve medications and surgery.

Some patients with UC only have disease in the rectum (proctitis). Others with UC have disease limited to the rectum and the adjacent left colon (proctosigmoiditis). Yet others have UC of the entire colon (universal IBD). Symptoms of UC are generally more severe with more extensive disease (larger portion of the colon involved with disease). The prognosis for patients with disease limited to the rectum (proctitis) or UC limited to the end of the left colon (proctosigmoiditis) is better than that of full colon UC. In patients with more extensive disease, blood loss from the inflamed intestines can lead to anemia, and may require treatment with iron supplements or even blood transfusions.

Rarely, the colon can acutely dilate to a large size when the inflammation becomes very severe. This condition is called toxic megacolon. Patients with toxic megacolon are extremely ill with fever, abdominal pain and distention, dehydration, and malnutrition. Unless the patient improves rapidly with medication, surgery is usually necessary to prevent colon rupture.

CD can occur in all regions of the gastrointestinal tract. With this disease intestinal obstruction due to inflammation and fibrosis occurs in a large number of patients. Granulomas and fistula formation are frequent complications of CD. Disease progression consequences include intravenous feeding, surgery and colostomy.

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids. Similarly, an “isolated” cell, such as a dendritic cell, has been substantially separated, produced apart from, or purified away from other cells of the organism in which the cell naturally occurs. Isolated cells can be, for example, at least 99%, at least 98%, at least 95%, at least 90%, at least 85%, or at least 80% pure.

Pharmaceutically acceptable vehicles: The pharmaceutically acceptable carriers (vehicles) useful in this disclosure are conventional. Remington's Pharmaceutical Sciences, by E. W. Martin, Mack Publishing Co., Easton, Pa., 19th Edition (1995), describes compositions and formulations suitable for pharmaceutical delivery of one or more therapeutic compounds, molecules or agents. In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Regulatory B cells (Bregs): A type of B cells that have suppressive regulatory function resulting in poor T-cell proliferation, T-cell activation, B-cell proliferation, B-cell activation. Surface markers and chemokine profiles characteristic of regulatory B cells, as well as subsets of regulatory B cells (e.g., IL-10 producing Bregs) are known to those of skill in the art (e.g., as described in DiLillo et al., Ann N.Y. Acad. Sci. 1183 (2010) 38-57, ISSN 0077-8923; the entire contents of which are incorporated herein by reference). In some embodiments, the presence of regulatory B cells can be determined by intracellular staining for IL-10 by flow cytometry. For example, after treatment B cells can be stained for surface markers, then fixed and permeabilized and stained for intracellular IL-10 and analyzed by flow cytometry.

Sequence identity/similarity: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. This homology is more significant when the orthologous proteins or cDNAs are derived from species which are more closely related (such as human and mouse sequences), compared to species more distantly related (such as human and C. elegans sequences).

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI website.

BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (1166÷1554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15÷20*100=75).

For comparisons of amino acid sequences of greater than about 30 amino acids, the Blast 2 sequences function is employed using the default BLOSUM62 matrix set to default parameters, (gap existence cost of 11, and a per residue gap cost of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity with the proteins listed in Table 6 or Table 7.

When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity with the proteins listed in Table 6 or Table 7. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. Such homologous nucleic acid sequences can, for example, possess at least about 60%, 70%, 80%, 90%, 95%, 98%, or 99% sequence identity with the genes listed in Table 6 or Table 7 as determined by this method. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Small interfering RNA (siRNA): A double-stranded nucleic acid molecule that modulates gene expression through the RNAi pathway (see, for example, Bass, Nature 411:428-9, 2001; Elbashir et al., Nature 411:494-8, 2001; and PCT Publication Nos. WO 00/44895; WO 01/36646; WO 99/32619; WO 00/01846; WO 01/29058; WO 99/07409; and WO 00/44914). siRNA molecules are generally 20-25 nucleotides in length with 2-nucleotide overhangs on each 3′ end. However, siRNAs can also be blunt ended. Generally, one strand of a siRNA molecule is at least partially complementary to a target nucleic acid, such as a target mRNA. siRNAs are also referred to as “small inhibitory RNAs,” “small interfering RNAs” or “short inhibitory RNAs.” As used herein, siRNA molecules need not be limited to those molecules containing only RNA, but further encompasses chemically modified nucleotides and non-nucleotides having RNAi capacity or activity. In an example, a siRNA molecule is one that reduces or inhibits the biological activity or expression of a gene product.

Stabilization: Modification of a nucleic acid, such as, but not limited to, an antisense molecule, to increase the half-life of the molecule. In some embodiments, chemically modified oligonucleotides can be included in an antisense molecule in order to stabilize the molecule. To increase stabilization, unnatural bases can be included, the sugars can be modified (such as at the 2′ position of the ribose), or the phosphate backbone can be modified. In some examples, phosphorothioate oligodeoxynucleotides can be included in the nucleic acid molecule, wherein one of the non-bridging oxygen atoms in the phosphodiester bond is replaced by sulfur. Additional forms of stabilized RNA is 1′-O-methyl RNA and 2′-O-methoxyethl RNA. Additional nucleic acid analogs that can be used for stabilization are peptide nucleic acids (PNAs), N3′5-phosphoroamidate (NP), 2′-fluoro-arabino nucleic acid (FANA), locked nucleic acid (LNA), mopholino phosphoroamidate (MF), cyclohexene nucleic acid (CeNA) and tri-cyclo DNA (tcDNA), see Kurreck, Eur. J. Biochem. 270: 1628-1644, 2003.

Subject: Animals, including warm blooded mammals such as humans and primates; avians; veterinary subjects, including domestic household or farm animals such as cats, dogs, sheep, goats, cattle, horses and pigs; laboratory animals such as mice, rats and guinea pigs; fish; reptiles; zoo and wild animals; and the like.

Treating or Treatment: A therapeutic intervention that reduces a sign or symptom of a disease or pathological condition related to a disease (such as IBD). Treatment can also induce remission or cure of a condition, such as IBD. In particular examples, a treatment results in preventing or reducing an IBD, for example by inhibiting the full development of an IBD. Prevention can occur, for example in a person who is known to have a predisposition to a disease such as an IBD, such as UC or CD. An example of a person with a known predisposition is someone with a history of the IBD in the family, or who has been exposed to factors or has genetic markers that predispose the subject to the IBD.

Reducing a sign or symptom associated with an IBD can be evidenced, for example, by a delayed onset of clinical symptoms of the disease in a susceptible subject, a reduction in severity of some or all clinical symptoms of the disease, a slower progression of the disease, a reduction in the number of relapses of the disease, an improvement in the overall health or well-being of the subject, or by other parameters well known in the art that are specific to the particular IBD.

Tolerogenic immune response: Any immune response that can lead to immune suppression specific to an antigen, cell, tissue, or organ. Such immune responses include any reduction, delay or inhibition in an undesired immune response specific to the antigen, cell, tissue, or organ. Such immune responses also include any stimulation, production, induction, promotion or recruitment in a desired immune response specific to the antigen or cell, tissue, organ. Tolerogenic immune responses, therefore, include the absence of or reduction in an undesired immune response that can be mediated by antigen reactive cells or tissue reactive cells as well as the presence or promotion of suppressive cells. Tolerogenic immune responses as provided herein include immunological tolerance.

Tolerogenic immune responses include any reduction, delay or inhibition in CD4+ T cell, CD8+ T cell or B cell proliferation and/or activity. Tolerogenic immune responses also include a reduction in antigen-specific antibody production. Tolerogenic immune responses can also include any response that leads to the stimulation, induction, production or recruitment of regulatory cells, such as CD4+ regulatory T cells (Treg) cells, CD8+Treg cells, Breg cells, etc. In some embodiments, the tolerogenic immune response is one that results in the conversion to a regulatory phenotype characterized by the production, induction, stimulation or recruitment of regulatory cells. Tolerogenic immune responses also include any response that leads to the stimulation, production or recruitment of CD4+Treg cells and/or CD8+Treg cells.

CD4+Treg cells can express the transcription factor FoxP3 and inhibit inflammatory responses and auto-immune inflammatory diseases (Human regulatory T cells in autoimmune diseases. Cvetanovich et al., Curr Opin Hematol. 2009 July; 16(4):274-9). Such cells also suppress T-cell help to B-cells and induce tolerance to both self and foreign antigens (Miyara et al., Allergy Clin Immunol. 2009 April; 123(4):749-55). CD4+Treg cells recognize antigen when presented by Class II proteins on APCs. CD8+Treg cells, which recognize antigen presented by Class I (and Qa-1), can also suppress T-cell help to B-cells and result in activation of antigen-specific suppression inducing tolerance to both self and foreign antigens. Disruption of the interaction of Qa-1 with CD8+Treg cells has been shown to dysregulate immune responses and results in the development of auto-antibody formation and an auto-immune lethal systemic-lupus-erythematosus (Kim et al., Nature. 2010 Sep. 16, 467 (7313): 328-32). CD8+Treg cells have also been shown to inhibit models of autoimmune inflammatory diseases including rheumatoid arthritis and colitis (CD4+CD25+ regulatory T cells in autoimmune arthritis. Oh et al., Curr Opin Gastroenterol. 2008 November; 24(6):733-41). In some embodiments, the compositions provided can effectively result in both types of responses (CD4+Treg and CD8+Treg). In other embodiments, FoxP3 can be induced in other immune cells, such as macrophages, iNKT cells, etc., the compositions provided herein can result in one or more of these responses as well.

Tolerogenic immune responses also include, but are not limited to, the induction of regulatory cytokines, such as cytokines that suppress the proliferation of T-cells and/or B-cells, Treg-produced cytokines; induction of inhibitory cytokines; the inhibition of inflammatory cytokines (e.g., interleuckin (IL)-4, IL-1b, IL-5, tumor necrosis factor (TNF)-α, IL-6, granulocyte macrophage colony stimulating factor (GM-CSF), interferon (IFN)-γ, IL-2, IL-9, IL-12p70 IL-17, IL-18, IL-21, IL-22, IL-23, macrophage colony stimulating factor (M-CSF), C reactive protein, acute phase protein, chemokines (e.g., MCP-1, RANTES, MIP-1α, MIP-1β, MIG, ITAC or IP-10), the production of anti-inflammatory cytokines (e.g., IL-4, IL-13, IL-10, IL-12p40, etc.), proteases (e.g., MMP-3, MMP-9), leukotrienes (e.g., CysLT-1, CysLT-2), prostaglandins (e.g., PGE2) or histamines; the inhibition of polarization to a Th17, Th1 or Th2 immune response; the inhibition of effector cell-specific cytokines: Th17 (e.g., IL-17, IL-25), Th1 (IFN-.gamma.), Th2 (e.g., IL-4, IL-13); the inhibition of Th1-, Th2- or Th17-specific transcription factors; the inhibition of proliferation of effector T cells; the induction of apoptosis of effector T cells; the induction of tolerogenic dendritic cell-specific genes; the induction of FoxP3 expression; the inhibition of antibody responses (e.g., antigen-specific antibody production); the inhibition of T helper cell response; and the production of TGF-β and/or IL-10; the inhibition of effector function of autoantibodies (e.g., inhibition in the depletion of cells, cell or tissue damage or complement activation).

Under conditions sufficient for: A phrase that is used to describe any environment that permits a desired activity. In one example the desired activity is formation of an immune complex. In particular examples the desired activity is treatment or prevention of symptoms of an IBD.

Vector: Nucleic acid molecules of particular sequence can be incorporated into a vector that is then introduced into a host cell, thereby producing a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Vectors can be viral vectors, such as adenoviral, retroviral, or lentiviral vectors. Vectors can also be non-viral vectors, including any plasmid known to the art.

### Methods for Treating Inflammatory Bowel Disease (IBD)

Methods are provided herein for treating or preventing inflammation of the bowel, such is in a subject with an IBD. The IBD can be UC, CD, collagenous colitis lymphocytic colitis, ischemic colitis, diversion colitis, Behcet's disease, and indeterminate colitis.

In some embodiments, the methods include administering to a subject with an IBD, or at risk of developing an IBD, a therapeutically effective amount of an agent, such as a nucleic acid molecule, polypeptide, small molecule or other compound that is capable of inhibiting expression of one, two or all of CD40, CD80 and CD86. In some embodiments, the agent that inhibits expression of CD40, CD80 and/or CD86, is an antisense compound, such as an antisense oligonucleotide, siRNA or ribozyme specific for CD40, CD80 or CD86. In other embodiments, the agent is a combination of a CD40 antisense oligonucleotide, siRNA or ribozyme, a CD80 antisense oligonucleotide, siRNA or ribozyme, and a CD86 antisense oligonucleotide, siRNA or ribozyme. The agent can be microspheres that include agents that inhibit CD40, CD80 and CD86 expression, such as a combination of antisense nucleic acids specific for CD40, CD80 and CD86. The agent can also be tolerogenic dendritic cells that include agents that inhibit CD40, CD80 and CD86 expression, such as a combination of antisense nucleic acids specific for CD40, CD80 and CD86.

A therapeutically effective amount of a compound is an amount sufficient to result in a biological effect (such as alleviating or preventing one or more signs or symptoms of an IBD). In some examples, an agent can decrease or increase the expression level of a target RNA by a desired amount, for example by at least 2-fold, at least 3-fold, at least 4-fold, at least 5-fold, at least 6-fold, at least 8-fold, at least 10-fold, at least 15-fold, at least 20-fold, at least 30-fold or at least 40-fold relative to a control or reference value. These agents can be the microspheres disclosed herein, see also U.S. Pat. No. 8,389,493; U.S. Pat. No. 8,022,046, and U.S. Pat. No. 7,694,574, incorporated herein by reference.

One skilled in the art can readily determine a therapeutically effective amount of an agent to be administered to a given subject by taking into account several factors, such as the size and weight of the subject; the extent of disease progression; the age, health and sex of the subject; the route of administration; and whether the administration is regional or systemic. One skilled in the art can also readily determine an appropriate dosage regimen for administering to a subject an agent for the treatment of IBD. For example, the disclosed agents, such as microspheres, or tolerogenic dendritic cells, can be administered one hour, twelve hours, one day, two days, five days, one week, two weeks or one month apart.

The agent can be combined with one or a combination of medicaments/treatments known to be useful in the treatment of IBD such as, but not limited to, salicylic acid derivatives, sulfasalazine (Azulfadine), mesalamine (Asacol, Pentasa), immunosuppressants (Imuran, 6-MP, cyclosporine); methotrexate, tumor necrosis factor (TNF)-α inhibitors (REMICADE® and HUMIRA®); and corticosteroids (ENTOCORT® and prednisone). The agent can be combined with treatments (experimental) for ulcerative colitis, include aloe vera, butyrate, boswellia, probiotics, antibiotics, and nicotine.

Improvement in IBD encompasses a reduction in the severity, duration, or prevention of any one or more clinical IBD symptoms, such as, but not limited to, abdominal cramps and pain; bloody stool; diarrhea; urgency to have a bowel movement; fever; loss of appetite; weight loss; mucus in the stool; ulceration of the large intestine; and anemia (due to blood loss). Moreover, the therapy can result in a reduction in the severity, duration, and/or risk of developing complications of the IBD, a reduced risk of the IBD subject developing profuse bleeding from the ulcers; perforation (rupture) of the bowel; strictures and obstruction; fistulae (abnormal passage) and perianal disease; toxic megacolon (acute nonobstructive dilation of the colon); and malignancy (for example, colon cancer).

### Antisense Compositions

The antisense compounds of use in the methods disclosed herein include nucleic acid sequences that bind to and inhibit translation of ribonucleic acids encoding CD40, CD80 and CD86. Thus, a combination of an antisense compound specific for CD40, an antisense compound specific for CD80- and an antisense compound specific for CD86 can be utilized for the treatment of IBD. Any type of antisense compound that specifically binds to ribonucleic acid (RNA) that encodes CD40, CD80 and CD86 is contemplated for use. In some examples, the agent is an antisense compound selected from an antisense oligonucleotide, a small inhibitory (si)RNA, a short hairpin RNA (shRNA), or a ribozyme specific for an RNA that encodes CD40, CD80 or CD86. Methods of designing, preparing and using antisense compounds are within the abilities of one of skill in the art. Furthermore, sequences for CD40, CD80 and CD86 are publicly available.

Antisense compounds can be prepared by designing compounds that are complementary to, and specifically bind, the target nucleotide sequence. Antisense compounds need not be 100% complementary to the target nucleic acid molecule to specifically bind with the target nucleic acid molecule. For example, the antisense compound, or antisense strand of the compound if a double-stranded compound, can be at least 75%, at least 80%, at least 85%, at least 90%, at least 95%, at least 99% or 100% complementary to the selected target nucleic acid sequence. Methods of screening antisense compounds for specificity are well known in the art (see, for example, U.S. Patent Application Publication No. 2003-0228689).

Exemplary nucleic acid sequences encoding human CD40, CD80 and CD86 are provided below:

Human CD40 (SEQ ID NO: 1):
GCCAAGGCTG GGGCAGGGGA GTCAGCAGAG
GCCTCGCTCG GGCGCCCAGT GGTCCTGCCG
CCTGGTCTCA CCTCGCTATG GTTCGTCTGC
CTCTGCAGTG CGTCCTCTGG GGCTGCTTGC
TGACCGCTGT CCATCCAGAA CCACCCACTG
CATGCAGAGA AAAACAGTAC CTAATAAACA
GTCAGTGCTG TTCTTTGTGC CAGCCAGGAC
AGAAACTGGT GAGTGACTGC ACAGAGTTCA
CTGAAACGGA ATGCCTTCCT TGCGGTGAAA
GCGAATTCCT AGACACCTGG AACAGAGAGA
CACACTGCCA CCAGCACAAA TACTGCGACC
CCAACCTAGG GCTTCGGGTC CAGCAGAAGG
GCACCTCAGA AACAGACACC ATCTGCACCT
GTGAAGAAGG CTGGCACTGT ACGAGTGAGG
CCTGTGAGAG CTGTGTCCTG CACCGCTCAT
GCTCGCCCGG CTTTGGGGTC AAGCAGATTG
CTACAGGGGT TTCTGATACC ATCTGCGAGC
CCTGCCCAGT CGGCTTCTTC TCCAATGTGT
CATCTGCTTT CGAAAAATGT CACCCTTGGA
CAAGCTGTGA GACCAAAGAC CTGGTTGTGC
AACAGGCAGG CACAAACAAG ACTGATGTTG
TCTGTGGTCC CCAGGATCGG CTGAGAGCCC
TGGTGGTGAT CCCCATCATC TTCGGGATCC
TGTTTGCCAT CCTCTTGGTG CTGGTCTTTA
TCAAAAAGGT GGCCAAGAAG CCAACCAATA
AGGCCCCCCA CCCCAAGCAG GAACCCCAGG
AGATCAATTT TCCCGACGAT CTTCCTGGCT
CCAACACTGC TGCTCCAGTG CAGGAGACTT
TACATGGATG CCAACCGGTC ACCCAGGAGG
ATGGCAAAGA GAGTCGCATC TCAGTGCAGG
AGAGACAGTG AGGCTGCACC CACCCAGGAG
TGTGGCCACG TGGGCAAACA GGCAGTTGGC
CAGAGAGCCT GGTGCTGCTG CTGCTGTGGC
GTGAGGGTGA GGGGCTGGCA CTGACTGGGC
ATAGCTCCCC GCTTCTGCCT GCACCCCTGC
AGTTTGAGAC AGGAGACCTG GCACTGGATG
CAGAAACAGT TCACCTTGAA GAACCTCTCA
CTTCACCCTG GAGCCCATCC AGTCTCCCAA
CTTGTATTAA AGACAGAGGC AGAAGTTTGG
TGGTGGTGGT GTTGGGGTAT GGTTTAGTAA
TATCCACCAG ACCTTCCGAT CCAGCAGTTT
GGTGCCCAGA GAGGCATCAT GGTGGCTTCC
CTGCGCCCAG GAAGCCATAT ACACAGATGC
CCATTGCAGC ATTGTTTGTG ATAGTGAACA
ACTGGAAGCT GCTTAACTGT CCATCAGCAG
GAGACTGGCT AAATAAAATT AGAATATATT
TATACAACAG AATCTCAAAA ACACTGTTGA
GTAAGGAAAA AAAGGCATGC TGCTGAATGA
TGGGTATGGA ACTTTTTAAA AAAGTACATG
CTTTTATGTA TGTATATTGC CTATGGATAT
ATGTATAAAT ACAATATGCA TCATATATTG
ATATAACAAG GGTTCTGGAA GGGTACACAG
AAAACCCACA GCTCGAAGAG TGGTGACGTC
TGGGGTGGGG AAGAAGGGTC TGGGGG
CD80 (SEQ ID NO: 2):
GACAAGTACT GAGTGAACTC AAACCCTCTG
TAAAGTAACA GAAGTTAGAA GGGGAAATGT
CGCCTCTCTG AAGATTACCC AAAGAAAAAG
TGATTTGTCA TTGCTTTATA GACTGTAAGA
AGAGAACATC TCAGAAGTGG AGTCTTACCC
TGAAATCAAA GGATTTAAAG AAAAAGTGGA
ATTTTTCTTC AGCAAGCTGT GAAACTAAAT
CCACAACCTT TGGAGACCCA GGAACACCCT
CCAATCTCTG TGTGTTTTGT AAACATCACT
GGAGGGTCTT CTACGTGAGC AATTGGATTG
TCATCAGCCC TGCCTGTTTT GCACCTGGGA
AGTGCCCTGG TCTTACTTGG GTCCAAATTG
TTGGCTTTCA CTTTTGACCC TAAGCATCTG
AAGCCATGGG CCACACACGG AGGCAGGGAA
CATCACCATC CAAGTGTCCA TACCTCAATT
TCTTTCAGCT CTTGGTGCTG GCTGGTCTTT
CTCACTTCTG TTCAGGTGTT ATCCACGTGA
CCAAGGAAGT GAAAGAAGTG GCAACGCTGT
CCTGTGGTCA CAATGTTTCT GTTGAAGAGC
TGGCACAAAC TCGCATCTAC TGGCAAAAGG
AGAAGAAAAT GGTGCTGACT ATGATGTCTG
GGGACATGAA TATATGGCCC GAGTACAAGA
ACCGGACCAT CTTTGATATC ACTAATAACC
TCTCCATTGT GATCCTGGCT CTGCGCCCAT
CTGACGAGGG CACATACGAG TGTGTTGTTC
TGAAGTATGA AAAAGACGCT TTCAAGCGGG
AACACCTGGC TGAAGTGACG TTATCAGTCA
AAGCTGACTT CCCTACACCT AGTATATCTG
ACTTTGAAAT TCCAACTTCT AATATTAGAA
GGATAATTTG CTCAACCTCT GGAGGTTTTC
CAGAGCCTCA CCTCTCCTGG TTGGAAAATG
GAGAAGAATT AAATGCCATC AACACAACAG
TTTCCCAAGA TCCTGAAACT GAGCTCTATG
CTGTTAGCAG CAAACTGGAT TTCAATATGA
CAACCAACCA CAGCTTCATG TGTCTCATCA
AGTATGGACA TTTAAGAGTG AATCAGACCT
TCAACTGGAA TACAACCAAG CAAGAGCATT
TTCCTGATAA CCTGCTCCCA TCCTGGGCCA
TTACCTTAAT CTCAGTAAAT GGAATTTTTG
TGATATGCTG CCTGACCTAC TGCTTTGCCC
CAAGATGCAG AGAGAGAAGG AGGAATGAGA
GATTGAGAAG GGAAAGTGTA CGCCCTGTAT
AACAGTGTCC GCAGAAGCAA GGGGCTGAAA
AGATCTGAAG GTCCCACCTC CATTTGCAAT
TGACCTCTTC TGGGAACTTC CTCAGATGGA
CAAGATTACC CCACCTTGCC CTTTACGTAT
CTGCTCTTAG GTGCTTCTTC ACTTCAGTTG
CTTTGCAGGA AGTGTCTAGA GGAATATGGT
GGGCACAGAA GTAGCTCTGG TGACCTTGAT
CAAGGTGTTT TGAAATGCAG AATTCTTGAG
TTCTGGAAGG GACTTTAGAG AATACCAGTG
TTATTAATGA CAAAGGCACT GAGGCCCAGG
GAGGTGACCC GAATTATAAA GGCCAGCGCC
AGAACCCAGA TTTCCTAACT CTGGTGCTCT
TTCCCTTTAT CAGTTTGACT GTGGCCTGTT
AACTGGTATA TACATATATA TGTCAGGCAA
AGTGCTGCTG GAAGTAGAAT TTGTCCAATA
ACAGGTCAAC TTCAGAGACT ATCTGATTTC
CTAATGTCAG AGTAGAAGAT TTTATGCTGC
TGTTTACAAA AGCCCAATGT AATGCATAGG
AAGTATGGCA TGAACATCTT TAGGAGACTA
ATGGAAATAT TATTGGTGTT TACCCAGTAT
TCCATTTTTT TCATTGTGTT CTCTATTGCT
GCTCTCTCAC TCCCCCATGA GGTACAGCAG
AAAGGAGAAC TATCCAAAAC TAATTTCCTC
TGACATGTAA GACGAATGAT TTAGGTACGT
CAAAGCAGTA GTCAAGGAGG AAAGGGATAG
TCCAAAGACT TAACTGGTTC ATATTGGACT
GATAATCTCT TTAAATGGCT TTATGCTAGT
TTGACCTCAT TTGTAAAATA TTTATGAGAA
AGTTCTCATT TAAAATGAGA TCGTTGTTTA
CAGTGTATGT ACTAAGCAGT AAGCTATCTT
CAAATGTCTA AGGTAGTAAC TTTCCATAGG
GCCTCCTTAG ATCCCTAAGA TGGCTTTTTC
TCCTTGGTAT TTCTGGGTCT TTCTGACATC
AGCAGAGAAC TGGAAAGACA TAGCCAACTG
CTGTTCATGT TACTCATGAC TCCTTTCTCT
AAAACTGCCT TCCACAATTC ACTAGACCAG
AAGTGGACGC AACTTAAGCT GGGATAATCA
CATTATCATC TGAAAATCTG GAGTTGAACA
GCAAAAGAAG ACAACATTTC TCAAATGCAC
ATCTCATGGC AGCTAAGCCA CATGGCTGGG
ATTTAAAGCC TTTAGAGCCA GCCCATGGCT
TTAGCTACCT CACTATGCTG CTTCACAAAC
CTTGCTCCTG TGTAAAACTA TATTCTCAGT
GTAGGGCAGA GAGGTCTAAC ACCAACATAA
GGTACTAGCA GTGTTTCCCG TATTGACAGG
AATACTTAAC TCAATAATTC TTTTCTTTTC
CATTTAGTAA CAGTTGTGAT GACTATGTTT
CTATTCTAAG TAATTCCTGT ATTCTACAGC
AGATACTTTG TCAGCAATAC TAAGGGAAGA
AACAAAGTTG AACCGTTTCT TTAATAA
CD86 (SEQ ID NO: 3):
AGTCATTGCC GAGGAAGGCT TGCACAGGGT
GAAAGCTTTG CTTCTCTGCT GCTGTAACAG
GGACTAGCAC AGACACACGG ATGAGTGGGG
TCATTTCCAG ATATTAGGTC ACAGCAGAAG
CAGCCAAAAT GGATCCCCAG TGCACTATGG
GACTGAGTAA CATTCTCTTT GTGATGGCCT
TCCTGCTCTC TGCTAACTTC AGTCAACCTG
AAATAGTACC AATTTCTAAT ATAACAGAAA
ATGTGTACAT AAATTTGACC TGCTCATCTA
TACACGGTTA CCCAGAACCT AAGAAGATGA
GTGTTTTGCT AAGAACCAAG AATTCAACTA
TCGAGTATGA TGGTATTATG CAGAAATCTC
AAGATAATGT CACAGAACTG TACGACGTTT
CCATCAGCTT GTCTGTTTCA TTCCCTGATG
TTACGAGCAA TATGACCATC TTCTGTATTC
TGGAAACTGA CAAGACGCGG CTTTTATCTT
CACCTTTCTC TATAGAGCTT GAGGACCCTC
AGCCTCCCCC AGACCACATT CCTTGGATTA
CAGCTGTACT TCCAACAGTT ATTATATGTG
TGATGGTTTT CTGTCTAATT CTATGGAAAT
GGAAGAAGAA GAAGCGGCCT CGCAACTCTT
ATAAATGTGG AACCAACACA ATGGAGAGGG
AAGAGAGTGA ACAGACCAAG AAAAGAGAAA
AAATCCATAT ACCTGAAAGA TCTGATGAAG
CCCAGCGTGT TTTTAAAAGT TCGAAGACAT
CTTCATGCGA CAAAAGTGAT ACATGTTTTT
AATTAAAGAG TAAAGCCCAT ACAAGTATTC
ATTTTTTCTA CCCTTTCCTT TGTAAGTTCC
TGGGCAACCT TTTTGATTTC TTCCAGAAGG
CAAAAAGACA TTACCATGAG TAATAAGGGG
GCTCCAGGAC TCCCTCTAAG TGGAATAGCC
TCCCTGTAAC TCCAGCTCTG CTCCGTATGC
CAAGAGGAGA CTTTAATTCT CTTACTGCTT
CTTTTCACTT CAGAGCACAC TTATGGGCCA
AGCCCAGCTT AATGGCTCAT GACCTGGAAA
TAAAATTTAG GACCAATACC TCCTCCAGAT
CAGATTCTTC TCTTAATTTC ATAGATTGTG
TTTTTTTTTT AAATAGACCT CTCAATTTCT
GGAAAACTGC CTTTTATCTG CCCAGAATTC
TAAGCTGGTG CCCCACTGAA TTTTGTGTAC
CTGTGACTAA ACAACTACCT CCTCAGTCTG
GGTGGGACTT ATGTATTTAT GACCTTATAG
TGTTAATATC TTGAAACATA GAGATCTATG
TACTGTAATA GTGTGATTAC TATGCTCTAG
AGAAAAGTCT ACCCCTGCTA AGGAGTTCTC
ATCCCTCTGT CAGGGTCAGT AAGGAAAACG
GTGGCCTAGG GTACAGGCAA CAATGAGCAG
ACCAACCTAA ATTTGGGGAA ATTAGGAGAG
GCAGAGATAG AACCTGGAGC CACTTCTATC
TGGGCTGTTG CTAATATTGA GGAGGCTTGC
CCCACCCAAC AAGCCATAGT GGAGAGAACT
GAATAAACAG GAAAATGCCA GAGCTTGTGA
ACCCTGTTTC TCTTGAAGAA CTGACTAGTG
AGATGGCCTG GGGAAGCTGT GAAAGAACCA
AAAGAGATCA CAATACTCAA AAGAGAGAGA
GAGAGAAAAA AGAGAGATCT TGATCCACAG
AAATACATGA AATGTCTGGT CTGTCCACCC
CATCAACAAG TCTTGAAACA AGCAACAGAT
GGATAGTCTG TCCAAATGGA CATAAGACAG
ACAGCAGTTT CCCTGGTGGT CAGGGAGGGG
TTTTGGTGAT ACCCAAGTTA TTGGGATGTC
ATCTTCCTGG AAGCAGAGCT GGGGAGGGAG
AGCCATCACC TTGATAATGG GATGAATGGA
AGGAGGCTTA GGACTTTCCA CTCCTGGCTG
AGAGAGGAAG AGCTGCAACG GAATTAGGAA
GACCAAGACA CAGATCACCC GGGGCTTACT
TAGCCTACAG ATGTCCTACG GGAACGTGGG
CTGGCCCAGC ATAGGGCTAG CAAATTTGAG
TTGGATGATT GTTTTTGCTC AAGGCAACCA
GAGGAAACTT GCATACAGAG ACAGATATAC
TGGGAGAAAT GACTTTGAAA ACCTGGCTCT
AAGGTGGGAT CACTAAGGGA TGGGGCAGTC
TCTGCCCAAA CATAAAGAGA ACTCTGGGGA
GCCTGAGCCA CAAAAATGTT CCTTTATTTT
ATGTAAACCC TCAAGGGTTA TAGACTGCCA
TGCTAGACAA GCTTGTCCAT GTAATATTCC
CATGTTTTTA CCCTGCCCCT GCCTTGATTA
GACTCCTAGC ACCTGGCTAG TTTCTAACAT
GTTTTGTGCA GCACAGTTTT TAATAAATGC
TTGTTACATT CATTTAAAAA AAAAAAAAA

SEQ ID NOs: 4-6 encode mRNA for CD40, CD80 and CD86, respectively.

In some embodiments, the antisense compounds are antisense oligonucleotides. The antisense oligonucleotides can be any suitable length to allow for specific binding to the target and modulation of gene expression. The length of an antisense oligonucleotide can vary, but is typically about 15 to about 40 nucleotides, including 15, 16, 17, 18, 19, 20, 21, 22, 23, 24, 25, 26, 27, 28, 29, 30, 31, 32, 33, 34, 35, 36, 37, 38, 39 or 40 nucleotides. In some embodiments, the antisense oligonucleotides are about 20 to about 35 nucleotides in length. The antisense oligonucleotides can be DNA, RNA or analogs thereof. Furthermore, the oligonucleotides provided herein can be unmodified or can comprise one or more modifications, such as modified internucleoside linkages, modified sugar moieties, modified bases, or a combination thereof. Oligonucleotide modifications are described in detail below.

In other embodiments, the antisense compounds are siRNA molecules. siRNAs useful for the disclosed methods include short double-stranded RNA from about 17 nucleotides to about 30 nucleotides in length, preferably from about 20 to about 35 nucleotides in length, such as about 25 to about 32 nucleotides in length. The siRNAs are made up of a sense RNA strand and a complementary antisense RNA strand annealed together by standard Watson-Crick base-pairing interactions. The sense strand includes a nucleic acid sequence that is substantially identical to a nucleic acid sequence contained within the target CD40, CD80 or CD86 gene product. In some non-limiting examples, a siRNA nucleic acid sequence that is “substantially identical” to a target sequence is a nucleic acid sequence that is identical to the target sequence, or that differs from the target sequence by one, two or three nucleotides. The sense and antisense strands of the siRNA can either include two complementary, single-stranded RNA molecules, or can be a single molecule having two complementary portions (which are base-paired) separated a single-stranded “hairpin” region.

The siRNA can also be altered RNA that differs from naturally-occurring RNA by the addition, deletion, substitution and/or alteration of one or more nucleotides. Such alterations can include addition of non-nucleotide material, such as to one or both of the ends of the siRNA or to one or more internal nucleotides of the siRNA; modifications that make the siRNA resistant to nuclease digestion; or the substitution of one or more nucleotides in the siRNA with deoxyribonucleotides. One or both strands of the siRNA can also include a 3′ overhang. As used herein, a “3′ overhang” refers to at least one unpaired nucleotide extending from the 3′-end of a duplexed RNA strand. Thus, in certain embodiments, the siRNA includes at least one 3′ overhang of from 1 to about 6 nucleotides (which includes ribonucleotides or deoxyribonucleotides) in length, from 1 to about 5 nucleotides in length, from 1 to about 4 nucleotides in length, or from about 2 to about 4 nucleotides in length. In a particular embodiment, the 3′ overhang is present on both strands of the siRNA and is 2 nucleotides in length. For example, each strand of the siRNA can comprise 3′ overhangs of dithymidylic acid (“TT”) or diuridylic acid (“uu”).

In other embodiments, the antisense compound is a ribozyme. Ribozymes are nucleic acid molecules having a substrate binding region that is complementary to a contiguous nucleic acid sequence of a CD40, CD80 or CD86 gene product, and which is able to specifically cleave this gene product. The substrate binding region need not be 100% complementary to the target CD40, CD80 or CD86 gene product. For example, the substrate binding region can be, for example, at least about 50%, at least about 75%, at least about 85%, or at least about 95% complementary to a contiguous nucleic acid sequence in a CD40, CD80 or CD86 gene product. The enzymatic nucleic acids can also include modifications at the base, sugar, and/or phosphate groups.

Antisense compounds, such as antisense oligonucleotides, siRNAs and ribozymes, can be produced chemically or biologically, or can be expressed from a recombinant plasmid or viral vector. Exemplary methods for producing and testing antisense compounds are well known in the art (see, for example, U.S. Pat. Nos. 5,849,902 and 4,987,071; U.S. Patent Application Publication Nos. 2002/0173478 and 2004/0018176; Stein and Cheng, Science 261:1004, 1993; Werner and Uhlenbeck, Nucl. Acids Res. 23:2092-2096, 1995; Hammann et al., Antisense and Nucleic Acid Drug Dev. 9:25-31). The antisense oligonucleotides can specifically inhibit CD40, CD80 or CD86 mRNA expression by at least 10%, 20%, 30%, 40%, 50%, 55% 60%, 65%, 70%, 75%, 80%, 90% or 95% of that seen with vehicle treated controls i.e., cells exposed only to the transfection agent and the PBS vehicle, but not an antisense oligonucleotide.

In some examples, the antisense compounds that specifically bind to CD40, CD80 or CD86 RNAs, and inhibit expression, contain one or more modifications to enhance nuclease resistance and/or increase activity of the compound. Modified antisense compounds include those comprising modified backbones or non-natural internucleoside linkages. As defined herein, oligonucleotides having modified backbones include those that retain a phosphorus atom in the backbone and those that do not have a phosphorus atom in the backbone.

Examples of modified oligonucleotide backbones include, but are not limited to, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkyl-phosphonates, thionoalkylphosphotriesters, and boranophosphates having normal 3′-5′ linkages, 2′-5′ linked analogs of these, and those having inverted polarity wherein the adjacent pairs of the nucleoside units are linked 3′-5′ to 5′-3′ or 2′-5′ to 5′-2′. Representative U.S. patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; and 5,625,050.

Examples of modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439.

In some embodiments, both the sugar and the internucleoside linkage of the nucleotide units of the oligonucleotide or antisense compound are replaced with novel groups. One such modified compound is an oligonucleotide mimetic referred to as a peptide nucleic acid (PNA). In PNA compounds, the sugar-backbone of an oligonucleotide is replaced with an amide containing backbone, in particular an aminoethylglycine backbone. The bases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262. Further teaching of PNA compounds can be found in Nielsen et al. (Science 254, 1497-1500, 1991).

Modified oligonucleotides can also contain one or more substituted sugar moieties. In some examples, the oligonucleotides can comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In other embodiments, the antisense compounds comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one example, the modification includes 2′-methoxyethoxy (also known as 2′-O-(2-methoxyethyl) or 2′-MOE) (Martin et al., Helv. Chim. Acta., 78, 486-504, 1995). In other examples, the modification includes 2′-dimethylaminooxyethoxy (also known as 2′-DMAOE) or 2′-dimethylaminoethoxyethoxy (also known in the art as 2′-O-dimethylaminoethoxyethyl or 2′-DMAEOE).

Similar modifications can also be made at other positions of the compound. Antisense compounds can also have sugar mimetics such as cyclobutyl moieties in place of the pentofuranosyl sugar. Representative United States patents that teach the preparation of modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920.

Oligonucleotides can also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases, such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Further modified bases have been described (see, for example, U.S. Pat. No. 3,687,808; and Sanghvi, Y. S., Chapter 15, Antisense Research and Applications, pages 289-302, Crooke, S. T. and Lebleu, B., ed., CRC Press, 1993).

Certain of these modified bases are useful for increasing the binding affinity of antisense compounds. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. Representative U.S. patents that teach the preparation of modified bases include, but are not limited to, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; and 5,750,692.

Exemplary antisense compounds specific for CD40, CD80 and CD86, such as antisense oligonucleotides, ribozymes and siRNAs are disclosed above; any of these compounds can be used to produce tolerogenic dendritic cells. In some embodiments, the ODNs are stabilized, such as by thioation.

Exemplary antisense-oligonucleotides having the following sequences:

CD 40-AS:
(SEQ ID NO: 4)
5′C*AC* AG*C C*GA* GG*C* AA*A GA*C* AC*C A*T*G
C*AG* GG*C* A-3′
(SEQ ID NO: 5)
CD80-AS:
5′-G*GG* AA*A G*CC* AG*G A*AT* CT*A G*AG* CC*A
A*TG G*A-3′;
(SEQ ID NO: 6)
CD86-AS:
5′-T*GG* GT*G C*TT* CC*G T*AA* GT*T C*TG* GA*A
C*AC* G*T*C-3′

In the sequences shown above, the asterisk indicates thioation. However, one of skill in the art can readily produce antisense ODNs that are stabilized by thioation at other nucleotides.

Thus, in some embodiments, the ODN is stabilized at 1 to 20 nucleotides, such as 10 to 20 nucleotides, 15 to 20 nucleotides, or 14 to 17 nucleotides. The ODN can be stabilized at 11, 12, 13, 14, 15, 16 or 17 nucleotides.

The oligonucleotides can be conveniently and routinely made through solid phase synthesis. Equipment for such synthesis is sold by several vendors including Applied Biosystems.

Any other means for such synthesis may also be employed. Similar techniques can be used to prepare other oligonucleotides such as the phosphorothioates and alkylated derivatives, modified amidites and controlled-pore glass (CPG) products such as biotin, fluorescein, acridine or psoralen-modified amidites and/or CPG (available from Glen Research, Sterling Va.) to synthesize fluorescently labeled, biotinylated or other modified oligonucleotides such as cholesterol-modified oligonucleotides.

Antisense ODNs, including these exemplary ODNs, can be administered to a dendritic cell, either in vivo or in vitro, using any means known to those of skill in the art, including in microspheres, as discussed below.

Typically, the antisense oligonucleotides are present in pharmaceutical compositions and formulations as pharmaceutically acceptable salts, i.e., salts that retain the desired biological activity of the parent compound. Pharmaceutically acceptable salts include base addition salts that are formed with metals, for example, sodium, potassium, magnesium or calcium cations, or as organic amines, for example, chloroprocaine, choline, diethanolamine or ethylenediamine. Pharmaceutically acceptable salts also include organic or inorganic acid salts of amines, for example, hydrochlorides, acetates or phosphates. Other suitable pharmaceutically acceptable salts are well known to those skilled in the art. Pharmaceutically acceptable excipients are available in the art, and include those listed in various pharmacopoeias. See, e.g., USP, JP, EP, and BP, and Handbook of Pharmaceutical Additives, ed. Ash; Synapse Information Resources, Inc. 2002.

Compositions and formulations for administration in vivo and in vitro include sterile aqueous solutions or emulsions that may further contain buffers, diluents, carriers, preservatives, stabilizer and other excipients. Compositions and formulations for oral administration include tablets, capsules, gel capsules, dragees, powders, suspensions, emulsions, microemulsions or solutions. Typically, the compositions and formulations for oral administration further include binders, bulking agents, carriers, coloring agents, flavoring agents, surfactants, chelators, emulsifiers and other excipients. Surfactants include fatty acids, esters of fatty acids, bile acids and their salts.

The amount of the therapeutic that will be effective depends on the nature of the disorder or condition to be treated, as well as the stage of the disorder or condition. Effective amounts can be determined by standard clinical techniques. The precise dose to be employed in the formulation will also depend on the route of administration, and should be decided according to the judgment of the health care practitioner and each patient's circumstances. An example of such a dosage range is 0.1 to 200 mg/kg body weight in single or divided doses. Another example of a dosage range is 1.0 to 100 mg/kg body weight in single or divided doses. In some examples, an effective amount of the antisense compound that is administered to a subject can range from about 5 to about 3000 micrograms/kg of body weight, from about 700 to about 1000 micrograms/kg of body weight, or greater than about 1000 micrograms/kg of body weight.

In some embodiments, an antisense compound, such as, but not limited to, a shRNA, can be expressed from a vector. Suitable promoters for expression include, but are not limited to, the U6 or H1 RNA pol III promoter sequences, or a cytomegalovirus promoter. Selection of other suitable promoters is within the skill in the art. The recombinant viral vectors of the invention can also comprise inducible or regulatable promoters for expression of the antisense molecule.

Suitable viral vectors include, but are not limited to, adenovirus vectors, adeno-associated virus vectors, retroviral vectors, lentiviral vectors, herpesviral vectors, poxviral vectors, and the like. For example, adenovirus vectors can be first, second, third and/or fourth generation adenoviral vectors or gutless adenoviral vectors. Adenovirus vectors can be generated to very high titers of infectious particles; infect a great variety of cells; efficiently transfer genes to cells that are not dividing; and are seldom integrated in the host genome, which avoids the risk of cellular transformation by insertional mutagenesis (Zern and Kresinam, Hepatology 25(2), 484-491, 1997). Representative adenoviral vectors which can be used for the methods provided herein are described by Stratford-Perricaudet et al. (J. Clin. Invest. 90: 626-630, 1992); Graham and Prevec (In Methods in Molecular Biology: Gene Transfer and Expression Protocols 7: 109-128, 1991); and Barr et al. (Gene Therapy, 2:151-155, 1995).

Adeno-associated virus (AAV) vectors also are suitable for administration. Methods of generating AAV vectors, administration of AAV vectors and their use are well known in the art (see, for example, U.S. Pat. No. 6,951,753; U.S. Published Patent Application Nos. 2007/036757, 2006/205079, 2005/163756, 2005/002908; and PCT Publication Nos. WO 2005/116224 and WO 2006/119458).

Retrovirus, including lentivirus, vectors can also be used with the methods described herein. Lentiviruses include, but are not limited to, human immunodeficiency virus (such as HIV-1 and HIV-2), feline immunodeficiency virus, equine infectious anemia virus and simian immunodeficiency virus. Other retroviruses include, but are not limited to, human T-lymphotropic virus, simian T-lymphotropic virus, murine leukemia virus, bovine leukemia virus and feline leukemia virus. Methods of generating retrovirus and lentivirus vectors and their uses have been well described in the art (see, for example, U.S. Pat. Nos. 7,211,247; 6,979,568; 7,198,784; 6,783,977; and 4,980,289).

Suitable herpesvirus vectors can be derived from any one of a number of different types of herpesviruses, including, but not limited to, herpes simplex virus-1 (HSV-1), HSV-2 and herpesvirus saimiri. Recombinant herpesvirus vectors, their construction and uses are well described in the art (see, for example, U.S. Pat. Nos. 6,951,753; 6,379,6741 6,613,892; 6,692,955; 6,344,445; 6,319,703; and 6,261,552; and U.S. Patent Application Publication No. 2003/0083289).

### Tolerogenic Dendritic Cells (DCs)

Dendritice cells (DCs) are important in the initiation and regulation of immune responses, and are instrumental in the induction and maintenance of tolerance (Banchereau and Steinman, Nature 392:245-252 (1998); Thomson and Lu, Transplantation 68:1-8 (1999)). DC activation can be defined by two distinct processes, (1) maturation which involves the upregulation of major histocompatibility complex (MHC) and costimulatory molecules, and (2) survival which involves the rescue of DCs from immediate apoptosis after the withdrawal of growth factors (see Rescigno et al., J. Exp. Med. 188:2175-2180 (1998). The mature DC expresses high levels of MHC class II and costimulatory molecules. In contrast, DCs with tolerogenic properties express low levels of costimulatory molecules and induce antigen-specific specific hyporesponsiveness by triggering T cell apoptosis (see Lu et al., Transplantation 60:1539-1545 (1995)).

The methods disclosed herein relate to the ability to manipulate the activation/maturation state of DCs, and producing tolerogenic DCs using antisense compounds specific for CD40, CD80 and CD86, such as antisense oligonucleotides, ribozymes and siRNAs (see above). The tolerogenic DCs can be used to treat and/or prevent IBD, including ulcerative colitis, Crone's disease, collagenous colitis lymphocytic colitis, ischemic colitis, diversion colitis, Behcet's disease, and indeterminate colitis in a subject. Generally, the subject is a mammalian subject, such as a human or a veterinary subject.

In some embodiments, the tolerogenic dendritic cells at least one of the following properties i) capable of converting naive T cells to Foxp3+T regulatory cells ex vivo and/or in vivo (e.g., inducing expression of FoxP3 in the naive T cells); blocking the conversion of naive T-cells to TH17 T-cells; iii) capable of deleting effector T cells ex vivo and/or in vivo; iv) retain their tolerogenic phenotype upon stimulation with at least one TLR agonist ex vivo (and, in some embodiments, increase expression of costimulatory molecules in response to such stimulus); and/or v) do not transiently increase their oxygen consumption rate upon stimulation with at least one TLR agonist ex vivo; and/or vi) capable of converting B cells to regulatory B cells ex vivo and/or in vivo. In some embodiments, the itDCs have at least 2, at least 3, 4, or all 5 of the above properties.

The tolerogenic DCs are generally derived from mammalian DCs, obtained from donor mammals of the same or different species, or from an autologus source (i.e. they are from the host). Thus, DCs can be autologous, allogeneic, or xenogeneic. If the subject is allogeneic, it can be matched for major histocompatibility complex (MHC) genes with the subject of interest. A therapeutically effective amount of tolerogenic DCs can be administered to a subject to prevent and/or treat IBD. In some embodiments, DCs are isolated from a donor, and transplanted into a recipient. The donor and the recipient can be the same subject, and thus the cells can be autologous. The donor and the recipient can be from different subjects, and thus the cells can be allogeneic.

In some embodiments, the donor and recipient are from different species, and thus the cells are xenogeneic. The tolerogenic and viral vector-comprising tolerogenic DCs do not have to be derived from the same species as the host to be treated. For instance, DCs may be isolated from a baboon donor to produce the tolerogenic and viral vector-comprising tolerogenic DCs of the present invention and may be administered into a human host to enhance tolerogenicity therein (see Starzl, et al., Immunological Reviews 141:213 (1994), incorporated herein by reference).

The tissues from which DCs may be isolated to produce the tolerogenic DCs include, but are not limited to, liver, spleen, bone marrow, peripheral blood, thymus or lymph nodes. In one embodiment, the source of the DCs is bone marrow.

A starting population of cells comprising dendritic cells can be obtained using methods known in the art. Such a population may comprise myeloid dendritic cells (mDC), plasmacytoid dendritic cells (pDC), and/or dendritic cells generated in culture from monocytes (e.g., MO-DC, MDDC). In some embodiments, dendritic cells and/or dendritic cell precursors can also be derived from a mixed cell population containing such cells (e.g., from the circulation or from a tissue or organ).

In certain embodiments, the mixed cell population containing DC and/or dendritic cell precursors is enriched such that DC and/or dendritic cell precursors make up greater than 50% (e.g., 55%, 60%, 65%, 70%, 75%, 80%, 85%, 90%, 95%, 98%, 99%, 99.5%, 99.9% or more) of the cell population. In some embodiments, the dendritic cells described herein are isolated by separation from some or all non-dendritic cells in a cell population. In exemplary embodiments, cells can be isolated such that a starting population comprising dendritic cells and/or dendritic cell precursors contains at least 50% or more dendritic cells and/or dendritic cell precursors, such as a purity of 50%, 55%, 60%, 65%, 70%, 75%, 80%, 85%, 90%, 95%, 98%, 99%, 99.5%, 99.9% or more.

In some embodiments, dendritic cells can be isolated using the techniques described in Current Protocols in Immunology, Wiley Interscience, Nov. 19, 2009, or in Woo et al., Transplantation, 58:484 (1994), incorporated herein by reference. In some embodiments, dendritic cells can be purified using fluorescence-activated cell sorting for antigens present on their surface, e.g., CD11c in the case of certain dendritic cells. In additional embodiments, DCs present in a starting population of cells express CD11c. In some embodiments, DCs and/or dendritic cell precursors present in a starting population of cells express class II molecules. A starting population of cells may be monitored for expression of various cell surface markers (e.g., including CD11c) using techniques known in the art.

In some embodiments, the DCs of are treated with antisense compounds specific for CD40, CD80 and CD86, such as antisense oligonucleotides, ribozymes and siRNAs act to produce the tolerogenic dendritic cells. The DCs can be transformed with a vector, such as a lentiviral vector, adenoviral vector, or any of the vectors disclosed above, that encode antisense compounds specific for CD40, CD80 and CD86, such as antisense oligonucleotides, ribozymes, shRNA and siRNAs to produce the tolerogenic dendritic cells. Thus, the dendritic cells can “comprise” these antisense compounds by treating them with the antisense compounds or by expressing these compounds.

Isolating and propagating the mammalian DCs may be accomplished by any technique known to the skilled artisan. See, e.g., Inaba et al., J. Exp. Med. 176:1693-1702 (1992); Lu et al., Transplantation 60:1539-1545 (1995); and Lu et al., Transplantation 64:1808-1815 (1997); Woo et al., Transplantation 58:848 (1994), all incorporated herein by reference. For example, the mammalian DCs may be generated from precursors, isolated from a donor, see the examples section below. Once generated, the mammalian DCs may be propagated by any suitable cell culturing technique known in the art (see Inaba et al., J. Exp. Med. 176:1693-1702 (1992); Lu et al., Transplantation 60:1539-1545 (1995); and Lu et al., Transplantation 64:1808-1815 (1997), all incorporated herein by reference).

Methods are provided for treating and/or preventing IBD, or a symptom thereof, in a host comprising (a) propagating immature mammalian DCs from a donor, (b) incubating said DCs with an antisense compounds for CD40, CD80 and CD86, such as antisense, ribozymes or siRNAs for CD40, CD80 and CD86 under conditions wherein the DCs internalize antisense compounds, (c) culturing DCs, and (d) administering the ODN-comprising DCs to the mammalian host in an effective amount. The method may further comprise incubating the DCs in the presence of one or more cytokines, such as GM-CSF and interleuckin (IL)-4.

The method for enhancing tolerogenicity in a subject can include producing and administering the tolerogenic DCs. However, the tolerogenic dendritic cells can be produced separately. Methods for administering the tolerogenic of a mammalian subject include, but are not limited to, conventional and physiologically acceptable routes, such as, for example, oral, pulmonary, parenteral (intramuscular, intra-articular, intraperitoneal, intravenous (IV) or subcutaneous injection), inhalation (via a fine powder formulation or a fine mist, aerosol), transdermal, intradermal, nasal, vaginal, rectal, or sublingual routes of administration. The tolerogenic DC can be administered intravenously or subcutaneously.

The tolerogenic DCs can be administered with a carrier. Such carriers include any suitable physiological solution or dispersant or the like. The physiological solutions comprise any acceptable solution or dispersion media, such as saline or buffered saline. The composition can also include antibacterial and antifungal agents, isotonic and adsorption delaying agents, and the like. The carrier may further include one or more immunosuppressive agents in dosage unit form. The pharmaceutical composition can also include additional treatment agents, such as salicylic acid derivatives sulfasalazine (Azulfadine), mesalamine (Asacol, Pentasa), immunosuppressants (Imuran, 6-MP, cyclosporine); methotrexate, tumor necrosis factor (TNF)-α inhibitors (REMICADE® and HUMIRA®); and corticosteroids (ENTOCORT® and prednisone). Other treatments for ulcerative colitis include aloe vera, butyrate, boswellia, probiotics, antibiotics, and nicotine. In some examples, retinoic acid and/or a transforming growth factor (TGF), such as, but not limited to, TGF-α or TGF-β is administered to the subject.

Dosage of the tolerogenic DCs of the present invention to be administered in vivo is determined with reference to various parameters, including the species of the host, the age, weight and disease status of the subject. The dosage is preferably chosen so that administration causes an effective result, as measured by molecular or clinical assays, prolongation of foreign graft survival, and alleviation of a sign and/or symptom of the IBD, or prevention of occurrence or flare-up. Dosages may range from 1×10⁴ DCs to 1×10⁹ DCs per administration. In one embodiment, the dosage ranges from 5×10⁵ DCs to 5×10⁷ DCs. To achieve maximal therapeutic effect, several administrations may be required. Administration may be conducted, for example, daily, weekly, monthly or yearly depending on the alleviation of the symptoms of the disease. Administration can continue as long as necessary to alleviate the disease.

In some embodiments, a maintenance dose is administered to a subject after an initial administration has resulted in a tolerogenic response in the subject, for example to maintain the tolerogenic effect achieved after the initial dose, to prevent an undesired immune reaction in the subject, or to prevent the subject becoming a subject at risk of experiencing an undesired immune response or an undesired level of an immune response. In some embodiments, the maintenance dose is the same dose as the initial dose the subject received. In some embodiments, the maintenance dose is a lower dose than the initial dose. For example, in some embodiments, the maintenance dose is about ¾, about ⅔, about ½, about ⅓, about ¼, about ⅛, about 1/10, about 1/20, about 1/25, about 1/50, about 1/100, about 1/1,000, about 1/10,000, about 1/100,000, or about 1/1,000,000 of the initial dose.

Preferably, tolerogenic immune responses lead to the inhibition of the development, progression or pathology of the IBD, such as, but not limited to, UC and CD. In some embodiments, the reduction of an undesired immune response or generation of a tolerogenic immune response may be assessed by determining clinical endpoints, clinical efficacy, clinical symptoms, disease biomarkers and/or clinical scores. Undesired immune responses or tolerogenic immune responses can also be assessed with diagnostic tests to assess the presence or absence of IBD, such as, but not limited to, UC or CD. Undesired immune responses, such as abdominal pain, diarrhea, and rectal bleeding can further be assessed. The effect of the administration of the tolerogenic DCs can be assessed. In some embodiments, the assessment is performed more than once on the subject to determine that a desirable immune state is maintained in the subject.

### Microspheres

Microparticles, microspheres, and microcapsules are solid or semi-solid particles having a diameter of less than one millimeter, such as less than 100 microns, less than 90 microns, less than 80 microns, less, than 70 microns, less than 60 microns, such as about 40 to about 60 microns, such as about 50 microns, which can be formed of a variety of materials, including synthetic polymers, proteins, and polysaccharides. Microspheres have been used in many different applications, primarily separations, diagnostics, and drug delivery.

A number of different techniques can be used to make these microspheres from synthetic polymers, natural polymers, proteins and polysaccharides, including phase separation, solvent evaporation, emulsification, and spray drying. Generally the polymers form the supporting structure of these microspheres, and the drug of interest is incorporated into the polymer structure. Exemplary polymers used for the formation of microspheres include homopolymers and copolymers of lactic acid and glycolic acid (PLGA) as described in U.S. Pat. No. 5,213,812; U.S. Pat. No. 5,417,986; U.S. Pat. No. 4,530,840; U.S. Pat. No. 4,897,268; U.S. Pat. No. 5,075,109; U.S. Pat. No. 5,102,872; U.S. Pat. No. 5,384,133; U.S. Pat. No. 5,360,610, and European Patent Application Publication Number 248,531. Block copolymers such as tetronic 908 and poloxamer 407 as described in U.S. Pat. No. 4,904,479; and polyphosphazenes as described in U.S. Pat. No. 5,149,543. Microspheres produced using polymers exhibit a poor loading efficiency and are often only able to incorporate a small percentage of the drug of interest into the polymer structure. Therefore, substantial quantities of microspheres often must be administered to achieve a therapeutic effect.

Spherical beads or particles are commercially available. For example, antibodies conjugated to beads create relatively large particles specific for particular ligands. The large antibody-coated particles are routinely used to crosslink receptors on the surface of a cell for cellular activation, are bound to a solid phase for immunoaffinity purification, and may be used to deliver a therapeutic agent that is slowly released over time, using tissue or tumor-specific antibodies conjugated to the particles to target the agent to the desired site.

In making the microspheres that are used for treatment of inflammatory bowel disease (IBD), antisense (AS)-oligonucleotides are dissolved in aqueous solution and combined with water soluble polymer(s) and a polycation. The polycation can be poly-L-lysine or poly-L-ornithine.

In some non-limiting examples, the solution is incubated at about 60-70° C., cooled to about 23° C., and the excess polymer is removed. Microspheres are formed which contain AS-oligonucleotides.

As disclosed above, any antisense molecules can be used in the methods disclosed herein. Exemplary AS-oligonucleotides having the following sequences, wherein an asterisk indicates thioation:

CD 40-AS:
(SEQ ID NO: 4)
5′C*AC* AG*C C*GA* GG*C* AA*A GA*C* AC*C A*T*G
C*AG* GG*C* A-3′
CD80-AS:
(SEQ ID NO: 5)
5′-G*GG* AA*A G*CC* AG*G A*AT* CT*A G*AG* CC*A
A*TG G*A-3′;
CD86-AS:
(SEQ ID NO: 6)
5′-T*GG* GT*G C*TT* CC*G T*AA* GT*T C*TG* GA*A
C*AC* G*T*C-3′

It should be noted that other nucleotides within AS oligonucleotides can have thioation at other residues. In one non-limiting example, the microspheres include AS oligonucleotides for CD40, CD80 and CD86 at a ratio of about 1:1:1.

In some embodiments, the nucleic acids comprise between about 30 and about 100 weight percent of the microspheres, such as about 30, about 40, about 50, about 60, about 70, about 80, about 90, about 95, about 96, about 97, about 98 or about 99 percent by weight. In other embodiments the microspheres have an average particle size of not greater than about 50 microns. In some embodiments, the microspheres have an average particle size of less than 50, less than 45, less than 40, less than 35 microns or less than 30 microns. The microspheres can have a particle size of about 50, about 45, about 40, about 35 or about 30 microns. In some embodiments, the microspheres have an average particle size of 0.2 microns to 8 microns. In additional embodiments, the microspheres have an average particle size of 0.5 microns to 4 microns. In further embodiments, the microspheres have an average particle size of about 2 microns.

In some non-limiting examples, the microspheres are prepared as follows: An aqueous solution of the oligonucleotide mixture is prepared by combining aliquots from three oligonucleotide solutions, each solution containing one of these three types. A solution containing the three types of oligonucleotides is prepared. The solutions preferably contain about 10 mg/ml oligonucleotide. These are combined with aliquots of a 10 mg/ml stock solution of polycation solution at volumetric ratios of polycation:oligonucleotide of from about 1:1 to about 4:1. Polymer solutions of polyvinyl pyrrolidone and/or of polyethylene glycol are prepared and combined with the other solutions. Heating, cooling, centrifuging and washing multiple times provide an aqueous suspension which typically is frozen and lyophilized to form a dry powder of microspheres comprising oligonucleotide and polycation.

Microspheres are a viable non-viral delivery tool for plasmid DNA and antisense oligonucleotides and other nucleic acids. They allow for in vitro delivery of Beta-Galactosidase plasmid DNA in 3T3 fibroblast cells. The microspheres protect plasmid DNA from nuclease activity. High levels of Beta-Galactosidase activity are expressed following transfection with the microsphere formulations.

Microspheres containing the antisense oligonucleotides of interest down-regulate surface cell antigens CD40, CD80 and CD86 that are involved in the activation of the autoimmune reaction. This can be accomplished by subcutaneous injection to dendritic cells located under the skin. The DNA and oligonucleotide microspheres are effective transfection vehicles in vitro and in vivo, see for example, U.S. Pat. No. 8,389,493; U.S. Pat. No. 8,022,046, and U.S. Pat. No. 7,964,574, which are incorporated herein by reference.

Without being bound by theory, dendritic cells take up the oligonucleotide microspheres and suppress the expression of surface cell antigens CD40, CD80 and CD86. The antisense oligonucleotide microspheres effectively treat and/or prevent development of inflammatory bowel disease.

Thus, in some embodiments, methods are provided for decreasing inflammation of the bowel in a subject. These methods include administering to the subject a therapeutically effective amount of a microsphere composition, wherein microspheres in the microsphere composition include oligonucleotides that are antisense to, and bind to, and inhibit translation of ribonucleic acid molecules selected from the group consisting of CD40, CD80 and CD86 ribonucleic acid molecules, and combinations thereof. The composition is administered in an amount effective to ameliorate the symptoms of inflammation of the bowel in the subject. The subject can have an inflammatory bowel disease, such as CD or UC. The methods can include administration of a composition containing microspheres that include oligonucleotides that are antisense to and targeted to bind to CD40, CD80 and CD86 ribonucleic acid molecules, such as a composition administered as an injectable form. The microspheres can include oligonucleotides are antisense to and bind to CD40 ribonucleic acid molecules, oligonucleotides are antisense to and bind to CD80 ribonucleic acid molecules, and oligonucleotides are antisense to and bind to CD86 ribonucleic acid molecules at a ratio of 1:1:1.

In additional embodiments, method are provided for treating inflammatory bowel disease, such as UC or CD, that include administering to the subject a therapeutically effective amount of a composition comprising microspheres, wherein the microspheres include a first oligonucleotide that has a first antisense sequence that targets a ribonucleic acid encoding CD40, a second oligonucleotide that has a second antisense sequence that targets a ribonucleic acid encoding CD80, and a third oligonucleotide that has an antisense sequence that targets a ribonucleic acid encoding CD86. The first, second and third oligonucleotides reduce or suppress in vivo expression of CD40, CD80 and CD86, respectively. In addition, the first oligonucleotide, second oligonucleotide and third oligonucleotide include greater than about 30 weight percent of the microspheres, based on total weight of the microspheres, and wherein the microspheres having an average particle size of at least 0.2 microns and not greater than about 50 microns, therein treating the inflammatory bowel disease in the subject. In some non-limiting examples, the microspheres have an average particle size of 0.2 microns to 8 microns. In additional non-limiting examples, the microspheres have an average particle size of 0.5 microns to 4 microns. In further non-limiting examples, the microspheres have an average particle size of about 2 microns. In other non-limiting examples, the first oligonucleotide, the second oligonucleotide and the third oligonucleotide are greater than 60% by weight of the microspheres and/or the first oligonucleotide, the second oligonucleotide and the third oligonucleotide are thiolated.

In some embodiments, the microspheres further include a polycation. Such s poly-L-lysine or poly-L-ornithine. A composition including these microspheres can be formulated an injectable composition suitable for in vivo delivery, such as for subcutaneous administration.

Furthermore, additional treatment agents can be administered, such as salicylic acid derivatives sulfasalazine (Azulfadine), mesalamine (Asacol, Pentasa), immunosuppressants (Imuran, 6-MP, cyclosporine); methotrexate, tumor necrosis factor (TNF)-α inhibitors (REMICADE® and HUMIRA®); and corticosteroids (ENTOCORT® and prednisone). Other treatments for ulcerative colitis include aloe vera, butyrate, boswellia, probiotics, antibiotics, and nicotine. In some examples, retinoic acid and/or a transforming growth factor (TGF), such as, but not limited to, TGF-α or TGF-β is administered to the subject.

The disclosure is illustrated by the following non-limiting Examples.

### EXAMPLES

### Example 1

### Materials and Methods

Animals: Female C57BL/6J mice were purchased from Jackson Laboratories (Bar Harbor, Me.) and were used between the ages of 7-8 weeks. All mice were maintained in a specific pathogen-free environment

Generation of DC: DC were generated from bone marrow progenitors obtained from C57BL/6 mice in 6-day cultures with GM-CSF and IL-4 using previously-published protocols (Giannoukakis et al., Mol Ther 2000; 1:430-7.; Machen et al., J Immunol 2004; 173:4331-41) in the continuous presence or absence of a mixture of phosphorothioate antisense DNA targeting the primary transcripts of CD40, CD80 and CD86. Detailed methods of generation are known in the art (Machen et al., J Immunol 2004; 173:4331-41; Harnaha et al., Diabetes 2006; 55:158-70, incorporated herein by reference.

DC generated in cultures with only GM-CSF and IL-4 served as control DC (cDC). The DC generated in the presence of the antisense DNA are immunosuppressive and immunoregulatory (iDC).

DSS colitis/treatment of mice with DC: A standard DSS induction protocol was followed (Aharoni et al., J Pharmacol Exp Ther 2006; 318:68-78; Okayasu et al., Gastroenterology 1990; 98:694-702). Mice were randomly placed into three groups (n=4 mice per group; two independent study cohorts totalling n=8 mice per treatment group); control, cDC recipients and iDC recipients. Three days prior to exposure to DSS mice were injected with 2×10⁶ DC intraperitoneally (i.p.) in a minimal volume of sterile endotoxin-free PBS or the PBS vehicle as control. All mice were then switched to drinking water containing 3.5% DSS to which they had ad libitum access for five days. On day three of exposure to DSS, a second injection of 2×10⁶ cDC, iDC or PBS vehicle i.p. was administered. Mice were euthanized 7-10 days after the initiation of DSS exposure.

Measurements/Assessment of colitis: Mice were weighed on the day before DSS exposure and then every day thereafter until euthanasia. Colitis was assessed by weight loss, stool consistency, fecal blood and anal prolapse. Upon euthanasia, colons were harvested, flushed and fixed for histopathologic and immunofluorescence assessment. Concurrently, the mesenteric lymph nodes and spleen were collected, made into single cells in preparation for flow cytometric measurements of the frequencies of immune cell populations.

Flow cytometry: FACSCalibur/FACSAria with DIVA support (BD Biosciences) workstations with species-specific antibodies, non-overlapping fluorophores and appropriate isotype controls were used for flow-sorting and FACS analyses. Cells were antibody-stained either after pre-enrichment for specific populations over magnetic columns (Mlltenyi Biotec), or stained as freshly-isolated single cells from mesenteric lymph nodes or spleen in vitro.

To measure Tregs, a detection system was used that includes the FJK-16s Foxp3-specific antibody, CD4-FITC clone RM4-5 and CD25-APC clone PC61.5 (eBioscience). For B-cell population characterization and FACS analysis, the following antibodies were used (all from BD Biosciences): B220-Pacific Blue clone RA3-6B2, CD19-PE Cy7 clone 1D3, CD5-PerCP clone 53-7.3, and CD1d-FITC clone 1B1. IL-10-producing cells were identified and characterized as Bregs following positive selection along IL-10 surface adsorption using a commercial magnetic isolation method (Miltenyi Biotec product #130-090-435, Auburn Calif.). Characterization of these cells as Bregs was then confirmed using standard FACS with the B-cell antibodies listed above.

To measure retinoic acid-producing CD103+DC, stained single splenocytes or mesenteric lymph node cells were first with the ALDEFLUOR® reagent (StemCell Technologies, BC, Canada), a substrate of retinaldehyde dehydrogenase (ALDH), the rate-limiting enzyme for retinoic acid biosynthesis (Moreb et al., Chem Biol Interact 2011; Moreb et al., Cytometry B Clin Cytom 2007; 72:281-9. Subsequently, a CD103-specific antibody (clone 2E7, Biolegend, CA) was used for staning, and the frequency of CD103+ALDEFLUOR+ cells was measured by FACS.

Histology/immunocytochemistry: The colons of mice were cut into proximal, middle, and distal segments. After being fixed in 4% paraformaldehyde (Sigma-Aldrich, MO) for 3-4 hrs, colon tissues were transferred to 30% sucrose (Sigma-Aldrich, MO) overnight. Tissues were embedded in Tissue-Tek OCT (Fisher Chemicals, NJ) and 10 micron-thick frozen sections were cut. For H&E staining, frozen sections were dried at room temperature and staining was then conducted with a commercially-available kit (Frozen Section Staining Kit; Thermo Fisher Scientific, NJ). For immunofuorescence staining, frozen sections were triple-stained with combinations of the following primary antibodies: rabbit anti-retinoic acid receptor alpha (Santa Cruz Biotechnology, CA), goat anti-CD19 (Santa Cruz Biotechnology, CA), and rat anti-IL-10 (Biolegend, CA). They were then incubated with the following corresponding secondary antibodies: donkey anti-rabbit Alexa Fluor 488, donkey anti-goat Alexa Fluor 649 (Invitrogen, NY), and donkey anti-rat Cy3 (Jackson ImmunoResearch Labs, PA). Adjacent sections were double-stained with primary antibodies goat anti-Foxp3 (Santa Cruz Biotechnology, CA) and rabbit anti-CD25 (Santa Cruz Biotechnology, CA), followed by donkey anti-rabbit Alexa Fluor 488 and donkey anti-goat Cy3 (Jackson ImmunoResearch Labs, PA). The double-stained sections then underwent nuclear staining. Control sections were incubated with only the fluorescence-labeled secondary antibodies in the absence of any prior staining with the specific primary antibodies.

For H&E-based inflammation assessment, each colon segment was scored individually, and these scores were summed to reach a total score for the entire colon. Histologic scores were assigned to each segment as follows: 0, normal; 1, ulcer or cell infiltration limited to the mucosa; 2, ulcer or limited cell infiltration in the submucosa; 3, focal ulcer involving all layers of the colon; 4, multiple lesions involving all layers of the colon, or necrotizing ulcer larger than 3 mm in length. Thus the total possible histologic score is 12. Scoring was performed by a pathologist blinded to the treatment of the mouse.

### Example 2

### DC Prevent DSS-Induced Colitis

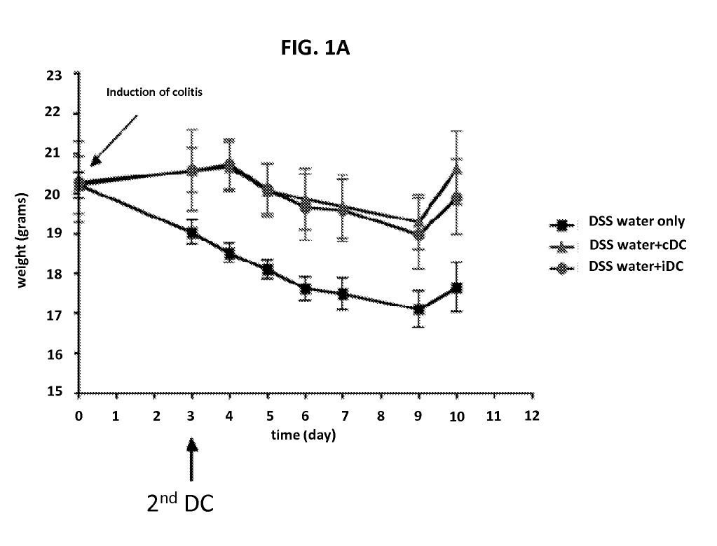

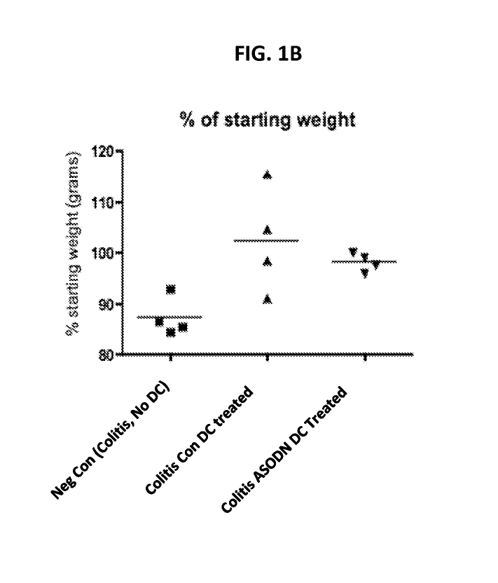

In FIG. 1A the median weights are shown as well as the range (error bars) of the mice in each of the three treatment groups. These observations were consistent among the two treatment cohorts which represented two independently-conducted experiments. Those mice that were not treated exhibited significant weight loss and typical symptoms associated with DSS colitis (evidence of blood in feces as well as anal prolapse). In contrast, the iDC and cDC treatments were effective in significantly-preventing weight loss. No evidence of blood was observed in stools in the DC-treated mice. In FIG. 1B, the weight at the end of the study period (7-10 days) as a % of starting weight is shown. cDC and iDC injection resulted in maintenance of the starting weight on a statistical basis.

### Example 3

### Colitis-Free DC Recipients Exhibit Increased Frequency of Foxp3+Tregs in the Spleen and the Mesenteric Lymph Nodes

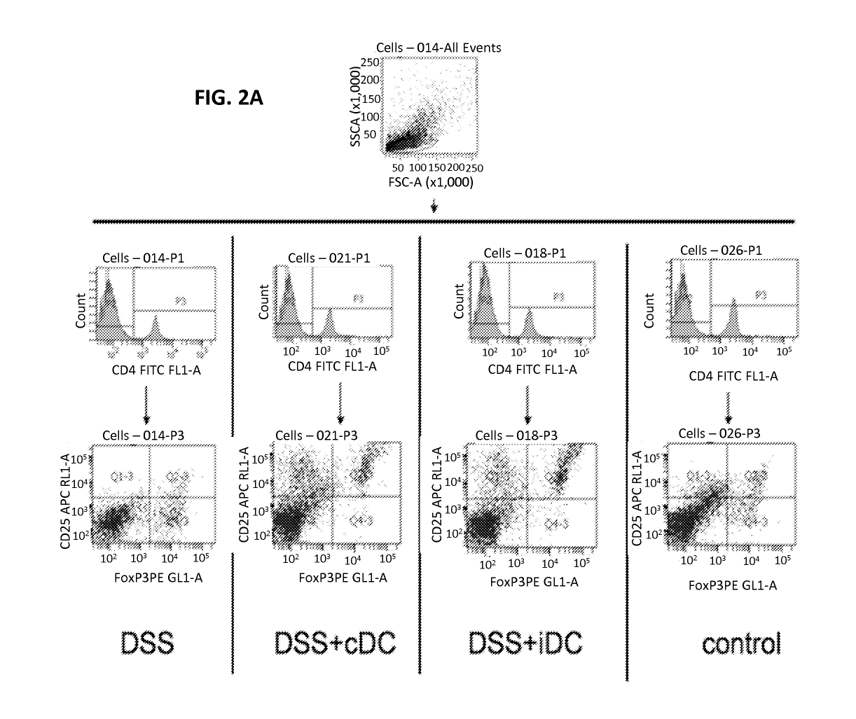

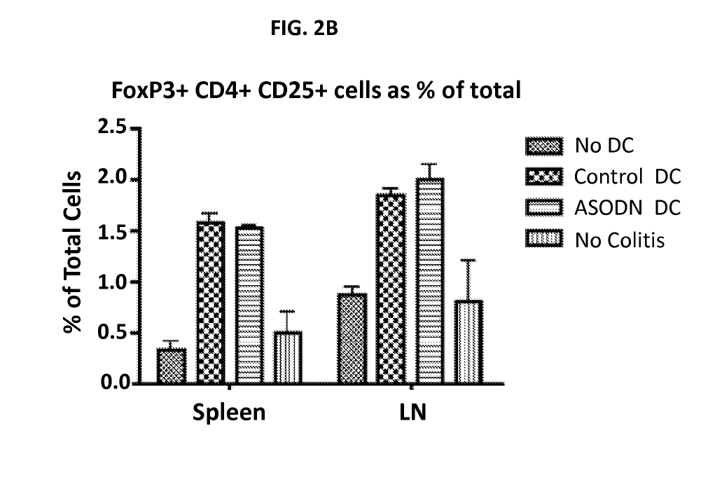

Given the evidence that Foxp3+Tregs are therapeutic for experimental colitis in mice, and that CD103+DC promote the differentiation of T-cells into Foxp3+Tregs while preventing conversion of gut T-cells into effector TH17-type cells (Coombes and Powrie, Nat Rev Immunol 2008; 8:435-46; Coombes et al., J Exp Med 2007; 204:1757-64; Annacker et al., J Exp Med 2005; 202:1051-61.), it was hypothesized that any beneficial outcome of the DC treatment would be associated with increased prevalence of Foxp3+Tregs in the mesenteric lymph nodes and possibly other lymphoid organs into which the exogenously-injected DC could potentially accumulate in. In FIG. 2, it is demonstrated that CD4+CD25+Foxp3+Tregs are increased in frequency as a % of total cells in spleen and in the mesenteric lymph nodes.

### Example 4

### Colitis-Free DC Recipients Exhibit Increased Frequency of B10 Bregs in the Spleen and the Mesenteric Lymph Nodes

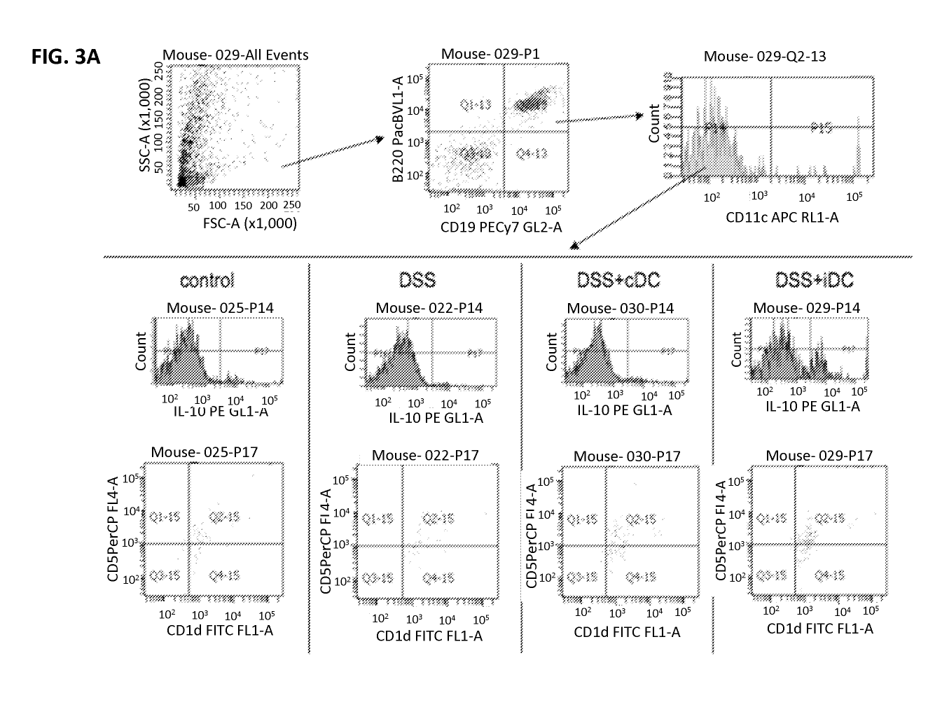

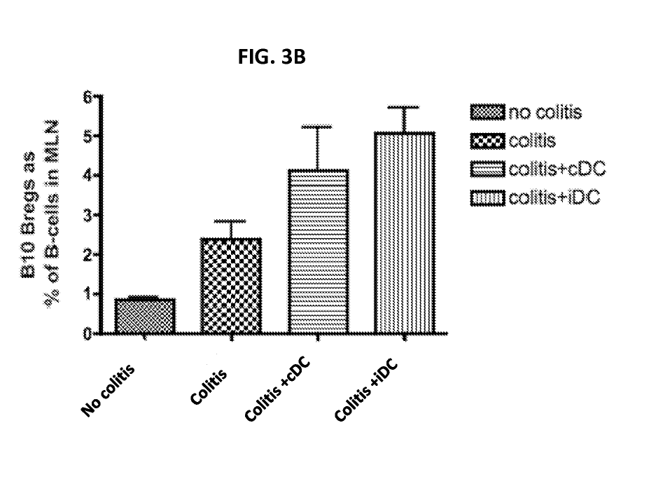

Accumulating data indicate that B-cells can act in a suppressive manner and two populations of these B-cells can transfer protection and improve experimental arthritis, lupus and colitis in mice (Mauri et al., Nat Rev Rheumatol 2010; 6:636-43; Yanaba et al., Immunol Rev 2008; 223:284-99; Yanaba et al., Immunity 2008; 28:639-50). Immature DC, including iDC, directly increased the prevalence of the “B10” regulatory B-cell population (Bregs) in vitro and in vivo. As an extension of these studies into experimental colitis, the frequency of B10 Bregs in the mesenteric lymph nodes and the spleen of mice pre-treated with cDC and iDC was measured prior to DSS colitis induction. In FIG. 3 it is shown that that B10 Bregs increased in frequency as a % of total B-cells (% of CD19+B220+ cells) in mesenteric lymph nodes, but not in spleen. In fact, DC treatment had no effect on the frequency of B10 Bregs in spleen of any treatment group, including DSS induction on its own.

### Example 5

### Colitis-Free DC Recipients Exhibited Increased Frequency of CD103+ALDEFLUOR®+DC In Vivo

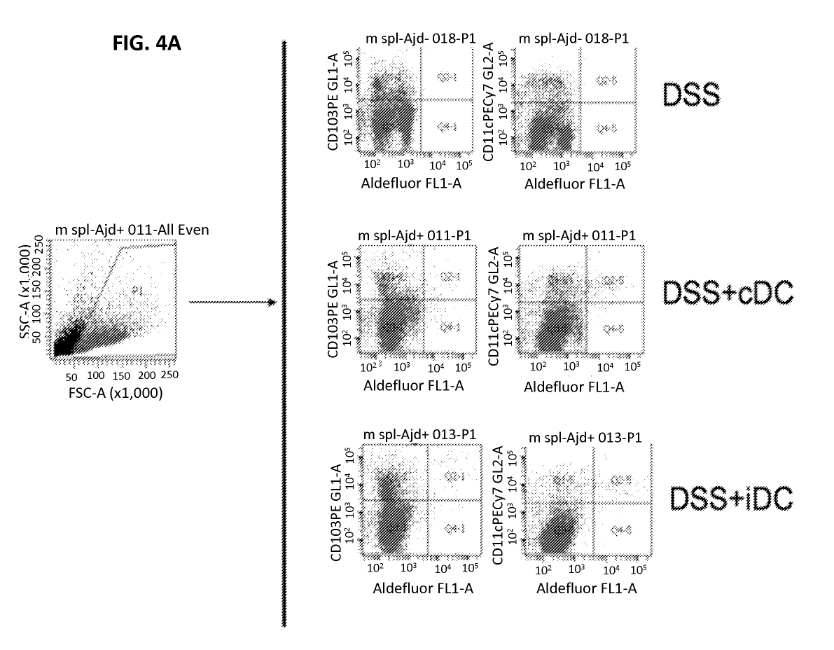

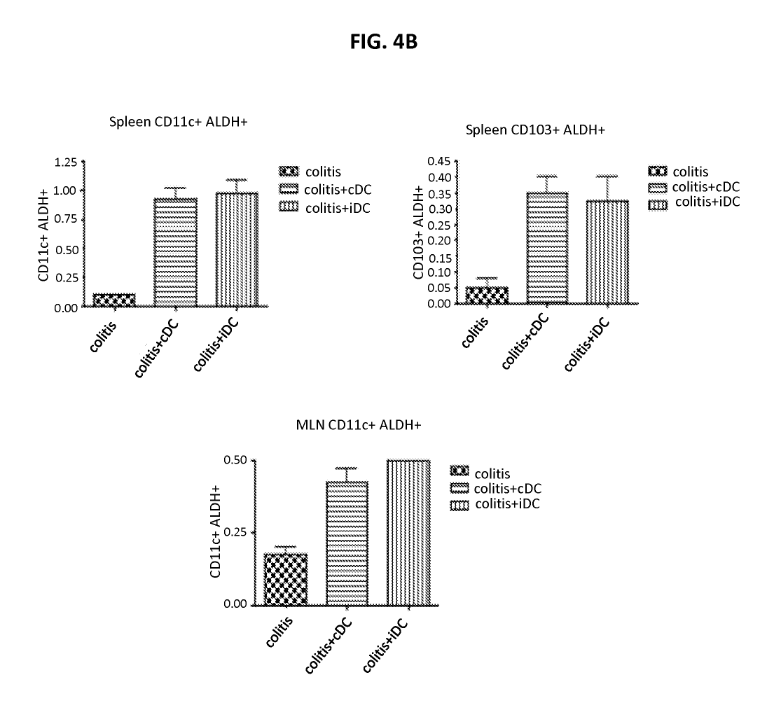

Although cDC and iDC express ALDH and produce retinoic acid in vitro but do not express CD103 on the cell surface, it was hypothesized that exogenous administration of these DC could change the endogenous DC phenotype in the spleen and the mesenteric lymph nodes of treated mice. The frequency of total DC expressing ALDH (CD11c+ALDEFLUOR®+) as well as the frequency of CD103+ALDEFLUOR®+ cells as a function of total splenocytes or mesenteric lymph node single cells was measured in DSS colitis mice treated with cDC or iDC prior to colitis induction and 3 days thereafter at the end of a 7-10 evaluation period. In FIG. 4, it is shown that splenic and mesenteric lymph node CD11c+ALDEFLUOR®+ cell frequency was significantly-increased in DC-recipients. However, the differences in CD103+ALDEFLUOR®+ cells between DC and control recipients were statistically-significant only in the splenic population (bottom graph, FIG. 4B), but not in the mesenteric lymph nodes.

### Example 6

### Colitis-Free DC Recipients Exhibit Inflammation-Attenuated Colon Architecture

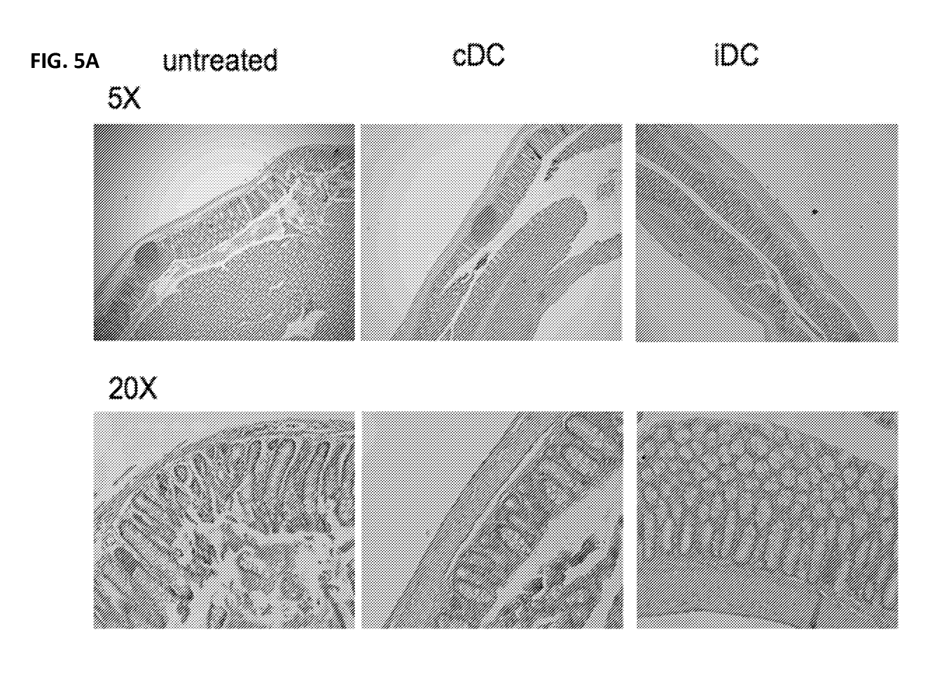

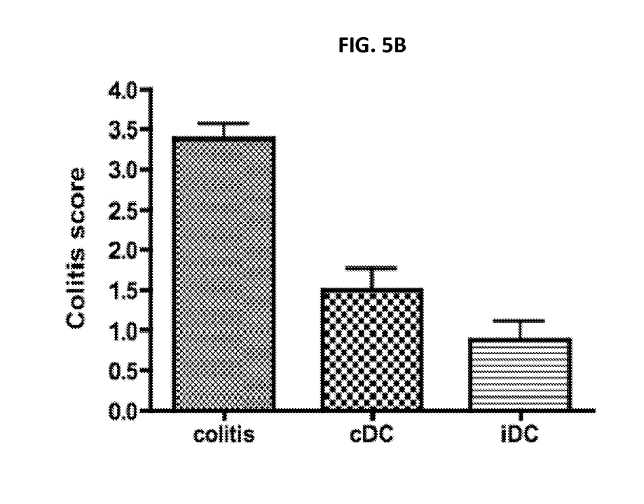

Even though the weight data and the regulatory immune cells suggested that DC treatment was beneficial, the degree of inflammation of the colons was ascertained. H&E staining of representative sections of tissue from control, cDC and iDC-treated mice suggested that DC significantly attenuated inflammation (FIG. 5A). In FIG. 5B, the scoring of inflammation in all treated mice is summarized.

### Example 7

### Colitis-Free DC Recipients Exhibit Increased Foxp3+ and CD19 Positivity Inside the Colon and Underlying Mucosa

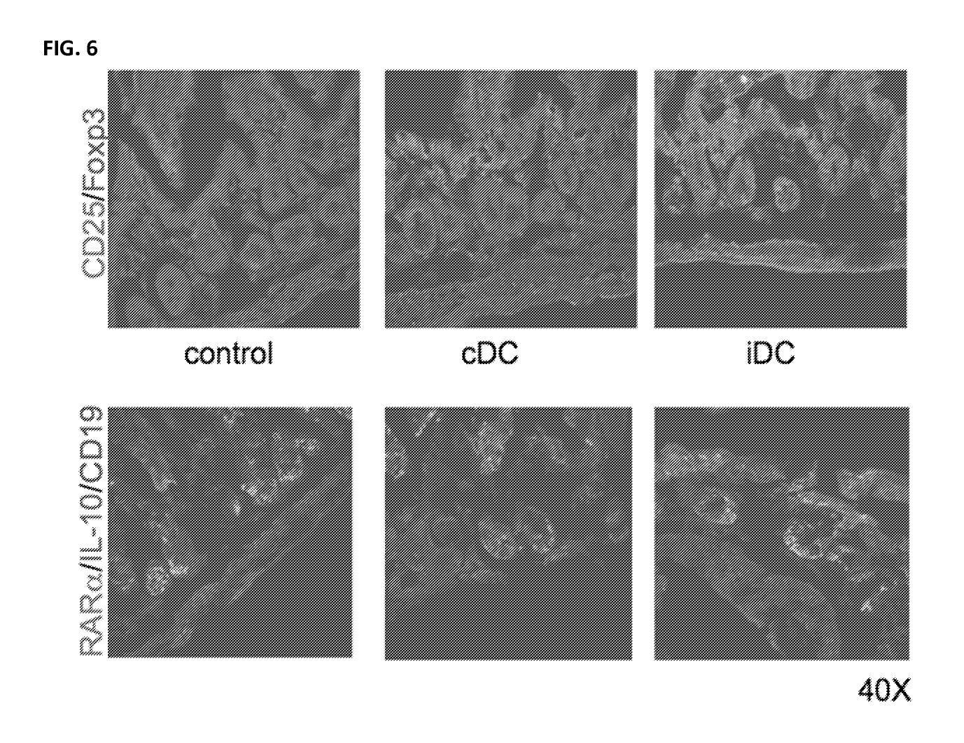

Given the increase in the Foxp3+Tregs as well as the B10 Bregs in the mesenteric lymph nodes of DC-treated mice, it was proposed that the substantial suppression of inflammation could be partly due to increased Treg and/or Breg presence in the colon mucosa and/or villi of DC-treated mice. The immunofluorescence microscopy data in FIG. 6 demonstrate an increase in CD19 staining suggestive of increased B-cell migration into the colon tissue of DC-treated mice. Additionally, a significant increase in immunoreactive Foxp3, especially in iDC recipients, was observed. Although these data do not formally demonstrate Breg or Treg accumulation as CD19 is a general B-cell marker and it was difficult to ascertain discrete overlap of Foxp3 and CD25 fluorescence, the data does support that DC promote an increase in B-cell accumulation co-ordinately with increased Foxp3 expression in the colon mucosa and villi. Although B10 Bregs produce IL-10, recent studies indicate that IL-10 expression is transient and thus more “mature” suppressive Bregs do not necessarily produce IL-10 or require it for suppression (Maseda et al., J Immunol 2012; 188:1036-48; Teichmann et al., J Immunol 2012; 188:678-85). This could account for the absence of any discernible IL-10 immunoreactivity in the excised colon tissue (lower panels of FIG. 6).

The data support the current model for how retinoic acid-producing DC could be therapeutic. It is demonstrated herein that cDC and iDC, two populations generated in vitro, produce retinoic acid, substantially attenuate the weight loss caused by DSS exposure and increase the frequency of Foxp3+Tregs in the mesenteric lymph nodes and spleen of DSS colitis mice. These studies also implicate B10 Bregs as responsive to cDC and iDC administration in vivo.

Previous studies have outlined two non-mutually exclusive pathways concerning Tregs. In the first, DC directly promote the proliferation of existing thymic-derived Foxp3-expressing Tregs inside the lymph nodes (Huang et al., J Immunol 2010; 185:5003-10; Azukizawa et al., Eur J Immunol 2011; 41:1420-34; Onoe et al., J Immunol 2011; 187:3895-903). However, a second mechanism appears to be operational in most instances of DC administration in vivo and involves the conversion of resting naive T-cells that either do not express, or express low levels of Foxp3, into suppressive Foxp3+Tregs (Josefowicz et al., Annu Rev Immunol 2012; 30:531-64). These adaptive, or induced, Tregs exhibit some plasticity in suppressive ability and depending on the presence or absence of cytokines like IL-10 or TGF-beta, can revert to non-suppressive cells (Josefowicz et al., supra). Retinoic acid and TGF-beta co-ordinately provide a third mechanism, effectively blocking the conversion of naive T-cells in the periphery into TH17-type cells and instead directing the T-cells into a potently-suppressive Foxp3+ population. This mechanism is prevalent in a number of IBD mouse models (Coombes et al., Nat Rev Immunol 2008; 8:435-46; Annacker et al., J Exp Med 2005; 202:1051-61; Coombes et al., Semin Immunol 2007; 19:116-26) and without being bound by theory may also be operational in the methods disclosed herein, especially since our DC produce retinoic acid. The data in FIG. 6 suggest that cDC and iDC (iDC>cDC) administration results in increased Foxp3 immunoreactvity through the entire colon tissue. Together with the increased splenic and mesenteric lymph node complement of Foxp3+Tregs, a significant tolerogenic state is established in vivo and this, along with the increase in Bregs, could be a powerful suppressant of the most acute and damaging experimental model of colitis; DSS.

Although B-cells have been traditionally-viewed as effector-type immune cells, mainly producing antibody and serving as accessory antigen-presenting cells, accumulating evidence supports a suppressive ability depending on the maturation and differentiation pathways selected during an immune response. IL-10 production appears to be a defining feature of immunosuppressive B-cells. More recently, two major populations of B-cells uniquely adapted to act as specific regulatory, immunosuppressive cells have been identified and characterized (Bouaziz et al., Curr Mol Med 2012; 12:519-2; Mauri and Bosma, Annu Rev Immunol 2012; 30:221-41; Fillatreau et al., at Immunol 2002; 3:944-50; Carter et al., Arthritis Res Ther 2012; 14:R32). Even though IL-10 expression is the main feature of these Bregs, its production is not a condition sine qua non for immunosuppression (Maseda et al., J Immunol 2012; 188:1036-48; Teichmann et al., J Immunol 2012; 188:678-85). Bregs, especially the B10 population suppress inflammation in experimental autoimmune encephalomyelitis, collagen-induced arthritis and colitis (Mizoguchi et al., Immunity 2002; 16:219-30; In a spontaneous model of murine colitis, the prevalence of B10 Bregs increases at the peak of inflammation and suppresses the disease by attenuating IL-1 and STAT3-mediated processes of immune reactivity (Mizoguchi et al., supra). In another model of colitis, in TCR-alpha-deficient transgenic mice, B-cell deficiency exacerbates disease and only CD40 ligand-activated B-cells can adoptively transfer protection and suppress the colitis inflammation (Mizoguchi et al., supra). Evidence suggests that B-cells isolated from mesenteric lymph nodes are stable suppressors of colitis, even though splenic marginal zone B-cell exhibit a plasticity of suppressive ability when adoptively co-transferred with G-alpha-i-2-deficient CD3+ T-cells into Rag2-deficient mice (Wei et al., Proc Natl Acad Sci USA 2005; 102:2010-5). Interestingly, in murine models of coltiis as well as in lupus, very few marginal zone splenic B-cells are found within the inflammation area further supporting a lymph node-source of suppressive B-cells. Without being bound by theory, the data presented herein are compatible with such a possibility; that stably-suppressive Bregs within the mesenteric lymph nodes are mobilized following their interaction with tolerogenic DC, or endogenous, intralymphatic recipient DC that differentiate into tolerogenic DC upon encounter with the exogenously-administered DC, in a retinoic acid-dependent manner. That Foxp3+Tregs and B10 Bregs are increased in frequency coordinately inside the mesenteric lymph node following cDC and iDC administration (which produce retinoic acid) indicates that DC are central in converting T-cells and B-cells into suppressive cells which then migrate into the inflamed colon structures to prevent or attenuate inflammation.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim all modifications and variations that fall within the scope and spirit of these claims.

1. A method for treating or preventing inflammatory bowel disease in a subject, comprising administering to the subject a therapeutically effective amount of a composition comprising an effective amount of tolerogenic dendritic cells, wherein the tolerogenic dendritic cells comprise at least one of an antisense compound specific for CD40, an antisense compound specific for CD80 and an antisense compound specific for CD86, thereby treating or preventing the inflammatory bowel disease in the subject.

2. The method of claim 1, wherein the dendritic cells are autologous to the subject.

3. The method of claim 1, wherein the subject is human.

4. The method of claim 1, wherein the inflammatory bowel disease is Crohn's disease.

5. The method of claim 1, wherein the inflammatory bowel disease is ulcerative colitis.

6. The method of claim 1, wherein the tolerogenic dendritic cells comprise the antisense compound that inhibits the expression of CD40, the antisense compound that inhibit the expression of CD80 and the antisense compound that inhibits the expression of CD86.

7. The method of claim 1, wherein administering the composition increases the number of regulatory T cells in the subject.

8. The method of claim 1, wherein administering the composition increases the number of regulatory B cells in the subject.

9. The method of claim 1, wherein administering the composition reduces inflammation of the subject's colon.

10. The method of claim 1, further comprising administering to the subject a therapeutically effective amount of a second agent.

11. The method of claim 10, wherein the second agent comprises sulfasalazin, mesalamine, an immunosuppressant, methotrexate, a tumor necrosis factor (TNF)-α inhibitor, retinoic acid, a transforming growth factor (TGF), or a corticosteroid.

12. The method of claim 6, wherein the antisense compound specific for CD40 comprises the nucleic acid sequence set forth as SEQ ID NO: 4, the antisense compound specific for CD80 comprises the nucleic acid sequence set forth as SEQ ID NO: 5, and the antisense compound specific for CD86 comprises the nucleic acid sequence set forth as SEQ ID NO: 6.

13. The method of claim 1, wherein the antisense compound specific for CD40, the antisense compound specific for CD80 and the antisense compound specific for CD86 are stabilized.

14. The method of claim 13, wherein the antisense compound specific for CD40, the antisense compound specific for CD80 and/or the antisense compound specific for CD86 are stabilized by thioatoin.

15-29. (canceled)
